(19) United States
(12) Patent Application Publication
(10) Pub. No.: US 2009/0028804 A1
(43) Pub. Date: Jan. 29, 2009

(54) AhR mediators

(75) Inventors: Jean Krutmann, Wegberg (DE); Martina Herrmann, Hameln (DE); Gabriele Vielhaber, Holzminden (DE); Jakob Ley, Holzminden (DE); Oskar Koch, Gottingen (DE)

Correspondence Address: CONNOLLY BOVE LODGE & HUTZ LLP, 1875 EYE STREET, N.W., SUITE 1100, WASHINGTON, DC, 20006, US

(73) Assignee: SYMRISE GmbH & Co. KG, Holzminden (DE)

(21) Appl. No.: 12/095,095

(22) PCT Filed: Nov. 28, 2006
(86) PCT No.: PCT/EP2006/069010

§ 371 Date: Sep. 5, 2008

Related U.S. Application Data
(60) Provisional application No. 60/796,854, filed on May 3, 2006.

(30) Foreign Application Priority Data
Nov. 28, 2005 (DE) 10 2005 056 890.4

(51) Int. Cl. A61K 8/33; C12Q 1/68; C40B 30/06; C40B 60/12; A61P 43/00; A61Q 17/04; C07D 311/22; C07D 307/78; A61K 31/352

(52) U.S. Cl. 424/59; 435/6; 506/10; 506/39; 549/403; 549/469; 514/456

(57) ABSTRACT

The invention relates to a method for finding and assessing agonists [and] antagonists of the aryl hydrocarbon receptor (Ah receptor; AhR), to the agonists and antagonists themselves and to uses thereof.

The invention relates to a method for finding and assessing agonists [and] antagonists of the aryl hydrocarbon receptor (Ah receptor; AhR), to the agonists and antagonists themselves and to uses thereof.

The skin is the human body's largest organ. Its most important function is to protect the body, on the one hand, from the uncontrolled escape of water and, on the other hand, from the penetration of harmful chemicals or bacteria and from solar radiation

If human skin is subjected to extended exposure to sunlight, this may result in many different kinds of harm. Examples which may be mentioned are sunburn, light-induced skin aging and skin cancer. This harmful action of sunlight is attributed among other things to the UVB radiation (280-320 nm) present in the spectrum of sunlight. In particular as a result of the recent sharp increase in the intensity of the UVB content in the spectrum of sunlight brought about by the ongoing destruction of the ozone layer, it is necessary to provide the skin with the most comprehensive protection possible against UVB radiation.

To provide protection from UV radiation, conventional sunscreen preparations contain substances for forming a protective layer on the skin which absorb and/or reflect radiation in the range from 280-400 nm (UV filters). Such photoprotective substances are for example inorganic oxides such as zinc oxide or organic UV absorbers such as for example derivatives of cinnamic acid or dibenzoylmethane. One disadvantage of these compounds, however, is that the protective layer they form can easily be destroyed by mechanical abrasion, water or detergents. It is therefore desirable, in addition to the above-mentioned UV filters, also to be able to make use of substances which exert a protective action within the skin.

If this problem is to be solved, it is vitally important to be aware of the molecular mechanisms by which UVB radiation is capable of having a harmful action on human skin. Investigations to this end have shown that the biological action of UVB radiation may, on the one hand, be attributed to the fact that UVB radiation brings about structural changes to the DNA molecules in the nucleus of skin cells. DNA repair enzymes are accordingly used for photoprotection (Stege et al. (2000) PNAS 97:1790).

On the other hand, it has been demonstrated that UVB radiation is capable of initiating changes at the level of the cell membrane which contribute to activation of growth receptors such as the epidermal growth factor receptor (EGF-R) and consequently to tumor formation (Ashida et al. (2003) Exp Dermatol 12:445; Lirvall et al. (1996) Biosci Rep 16:227). Such EGF-R activation can be inhibited by antioxidative enzymes (Lirvall et al. (1996) Biosci Rep 16:227).

UVB and UVA light also induce expression of cyclooxygenase-2 and matrix metalloproteinases (Pentland et al. (1999) Carcinogenesis 20(10):1939-44). Cyclooxygenases are among the key enzymes involved in the inflammatory response. They catalyze the first step of the synthesis of a series of inflammation mediators (prostaglandins, prostacyclins, thromboxanes) from arachidonic acid. There are 2 forms: cyclooxygenase-1 (COX-1) is the constitutive, permanently expressed form, while expression of COX-2 occurs only after stimulation by cellular signals, for example as a result of tissue damage or inflammation.

Matrix metalloproteinases (MMPs) are enzymes which are capable of proteolytically degrading the macromolecules of the extracellular matrix (ECM). MMPs have broad, often overlapping substrate specificity and, in combination, they are capable of breaking down all the protein components of the extracellular matrix. Around 20 MMPs have hitherto been identified. In human skin, a major role is played primarily by MMP-1 (collagenase-1), MMP-2 (gelatinase A), MMP-9 (gelatinase B) and MMP-3. Apart from cleaving collagen-1 and -3, MMP-1 also cleaves pro-MMP-2 and pro-MMP-9, so activating them. MMP-2 and MMP-9 are among the elastin-degrading proteases (A. Thibodeau, Cosmetics & Toiletries 2000, 115 (11), 75-82).

It has been established that old skin has a content of MMPs which distinctly higher than that of young skin (J. H. Chung et al., J. Invest. Dermatol, 2001, 117, 1218-1224). MMPs also play a decisive role in premature skin aging brought about by exogenous factors. A still further increased level of MMPs has been detected in light-aged skin relative to aged skin provided with light protection (J. H. Chung et al., J. Invest. Dermatol, 2001, 117, 1218-1224). Induction of matrix metalloproteinases has been demonstrated not only for UVA and UVB radiation, but also for infrared radiation. Such induction has been observed both in vitro in cultured human dermal fibroblasts and in vivo in UV-irradiated human skin. Stimulation with tobacco smoke also resulted in upregulation of MMP expression in human dermal fibroblasts.

It is furthermore frequently desired, in particular for cosmetic reasons, to influence the skin's tan, if possible without any harmful effects to skin cells occurring in so doing. In particular, skin tanning should also be achieved without exposure of the skin to be tanned to UVB radiation; it is likewise intended to allow skin lightening.

Attempts to achieve such a tan by purely cosmetic measures, in particular by applying topical tanning agents or skin-lightening agents such as creams, emulsions or lotions, have hitherto met with little success. On the one hand, pigment-containing preparations, as have long been used for makeup preparations, are conventionally offered as tanning agents. Problematic issues are that the tan produced in this way may readily be removed by mechanical action, for example when washing, and that the natural brown shade of healthy tanned skin can be achieved only with difficulty. “Self-tanning agents”, on the other hand, mainly involve using chemical oxidizing agents, such as for example dihydroxyacetone, as the active ingredient; this active ingredient only reacts with the proteins of the stratum corneum of human skin and, by oxidizing histidine and tryptophan stains the stratum corneum an orange-brown color. While the color produced by this oxidation is indeed less sensitive to mechanical action than makeup, it is often considered unnatural and dissimilar to the color of healthy tanned skin.

Furthermore, application of conventional tanning agents, in particular the described pigment preparations and self-tanning agents, provide the user with the unjustified feeling, thanks to the quickly and simply achieved staining effect on the skin, of being similarly protected as after the natural skin tanning which develops on exposure to sunlight. This erroneous idea almost automatically results in the acceptable sunlight exposure time being exceeded and so causes particularly serious skin damage.

Attempts have thus been made to stimulate the skin's synthesis of melanin without causing skin damage such as that which occurs on irradiation with UVB light. For example, it has been attempted to simulate the skin cells' “SOS response”, which is brought about by the known skin damage resulting from UV radiation and induces melanin formation, by stimulating melanocytes with certain pTpT oligonucleotides. The hope was, as a consequence, to increase the melanin concentration in the skin and so achieve not only a natural tan but also at the same time the associated and desired improvement in sun protection without any UV exposure. However, these investigations have led to virtually no usable results (cf. Eller et al. Nature 1994, vol. 372, page 414). Apart from the previously inadequate tests with oligonucleotides such as pTpT, no further substances have yet been found which might give rise to a better solution to the problem. The mechanisms by which new formation of melanin takes place or could take place better in the skin without the cells being irradiated and so inevitably damaged have hitherto remained unelucidated. The possible expression of aryl hydrocarbon receptors (AhR) by melanocytes has previously been reported only in connection with the formation and propagation of tumour cells under the influence of the extremely toxic dioxin derivative 2,3,7,8-tetrachlorodibenzo-p-dioxin (TCODD) (cf. Toxicol. Appl. Pharmacol of 24, Jun. 2005, cf. below). A person skilled in the art would not, however, be able to draw any conclusions from this which might result in the production of an agent for safe skin tanning. As a result, a very great need still remains for tanning agents which, without UVB irradiation and thus without radiation damage and without denaturing substances, which merely stain skin proteins, and by simple application onto the skin bring about an increase in melanin in the melanocytes and so, without further action, bring about increased sun protection and simultaneously a skin color which is as far as possible indistinguishable from a natural suntan.

On the other hand, many people have a need to lighten their naturally dark skin color or to prevent skin pigmentation. Very safe and effective skin- and hair-lightening agents are required for this purpose.

Skin-lightening active ingredients interact in some way with melanin metabolism or catabolism. Melanin pigments, which are generally brown to black in color, are formed by the melanocytes in the skin, are transferred into the keratinocytes and color the skin or hair. In mammals, brown-black eumelanins are mainly formed from hydroxy-substituted aromatic amino acids such as L-tyrosine and L-DOPA, while yellow to red pheomelanins are additionally formed from sulfur-containing molecules (Cosmetics & Toiletries 1996, 111 (5), 43-51). Starting from L-tyrosine, the key enzyme tyrosinase, which contains copper, forms L-3,4-dihydroxyphenylalanine (L-DOPA) which is turn converted by tyrosinase to dopachrome. Over several steps catalyzed by various enzymes, the latter is oxidized to form melanin.

Many skin- and hair-lightening agents contain tyrosinase inhibitors of greater or lesser strength. However, this is only one possible way to lighten skin and hair.

UV-absorbing substances are furthermore used to provide protection from the increase in skin pigmentation induced by UV light. However, this is a purely physical effect and must be distinguished from the biological action of skin-lightening agents on cellular melanin formation, which is also detectable in the absence of UV light. Moreover, UV absorbers do not bring about a true skin lightening effect, but instead merely prevent the increase in skin pigmentation induced by UV light.

Conventional commercial cosmetic or therapeutic skin- and hair-lightening formulations in particular make use of hydroquinone, hydroquinone derivatives, such as for example arbutin, vitamin C, derivatives of ascorbic acid such as for example ascorbyl palmitate, kojic acid and kojic acid derivatives such as for example kojic acid dipalmitate.

One of the most frequently used skin- and hair lightening agents is hydroquinone. However, this compound has a cytotoxic effect on melanocytes and an irritant action on the skin. As a result, such preparations are no longer admissible for cosmetic applications, for example in Europe, Japan and South Africa. Moreover, hydroquinone is very susceptible to oxidation and can be stabilized only with difficulty in cosmetic formulations.

Arbutin is a hydroquinone glucoside which is hydrolyzed in situ to form hydroquinone and is therefore just as toxicologically questionable as hydroquinone.

Vitamin C and ascorbic acid derivatives have an only inadequate action on the skin. In addition, they do not act directly as tyrosinase inhibitors, but instead reduce the colored intermediates in melanin biosynthesis.

Kojic acid (5-hydroxy-2-hydroxymethyl-4-pyranone) is a tyrosinase inhibitor which inhibits the enzyme's catalytic action by chelating its copper atoms; it is used in commercial skin- and hair-lightening agents, but has an elevated sensitizing potential and causes contact allergies.

It was an object of the present invention to overcome the disadvantages of the prior art and in particular to provide highly active skin-tanning and skin-lightening agents which should bring about maximally natural skin tanning or maximally effective prevention of skin tanning or skin lightening, in each case as far as possible without damaging skin cells.

It has now surprisingly been found that both skin tanning and skin lightening is achievable by influencing the aryl hydrocarbon receptor (AhR). The aryl hydrocarbon receptor (AhR) (NCBI gene accession number BC0700800) has previously merely been known as a central element in the detoxification of exogenous pollutants. The receptor mediates the biological response to polycyclic aromatic hydrocarbons (PAH) such as benzo[a]pyrene and halogenated PAH such as 2,3,7,8-tetrachlorodibenzo-p-dioxin (TCDD). AhR is a ligand-activated transcription factor which, once a ligand has bound, translocates into the cell nucleus. Here, it forms a dimer with a further transcription factor, the aryl hydrocarbon receptor nuclear translocator (ARNT), binds to regulatory gene sequences and induces transcription of various genes such as for example CYP1A1 and CYP1B1. Consequences of AhR activation include the development of skin tumors (Shimizu et al. (2000) 97:779), skin irritation and inflammation, the development of allergies, atopical dermatitis and itching and disruption of skin integrity (Tauchi et al. (2005) Mol. Cell Biol. 25: 9360-8; Henley et al., Arch. Biochem. Biophys. (2004) 422: 42-51) and induction of MMP-1 (collagenase-1) (Murphy et al. (2004) J. Biol. Chem. 279, 25284-2593).

UVB light induces CYP1A1 expression in human keratinocytes and lymphocytes and in the mouse hepatoma cell line Hepa-1 (Wei et al., Chem Biol Internet (1999) 118: 127-40). However, only for mouse Hepa-1 cells has it been proven that CYP1A1 induction is AhR-dependent. AhR activation is, however as explained further below, dependent on cell type, such that it is not possible to extrapolate from the action on mouse hepatoma cells to an action on human skin cells. Moreover, CYP1A1 may also be induced by pathways independent of AhR (Guigal et al. (2001) Life Sci.; 68(18):2141-50; Tijet et al. (2006), Mol Pharmacol 69(1): 140-153). No necessary connection has therefore been established between UVB-AhR-CYP1A1 in melanocytes and keratinocytes.

Stilbenes are known as Ah receptor ligands from WO 99/56737. While some of the stilbenes apparently do bind to the Ah receptor, they do not induce CYP1A1. These stilbenes include 3,4,3′,5-tetrahydroxystilbene or piceatannol, 2,3′,4,5′-tetrahydroxystilbene or oxyresveratrol and 3,5,4′-trihydroxystilbene or resveratrol, in particular trans-resveratrol. No photoprotective action, in particular against UVB radiation, is described. A disadvantageous feature of stilbenes is that they are photolabile and frequently bring about endocrine effects. For example, resveratrol is an antiandrogen (Mitchell et al. (1999) Cancer Res. 59: 5892-5895).

Henry et al (Mol Pharmacol 55 (1999):716-25) describe 3-methoxylated flavones bearing an electron-attracting substituent in position 4 as effective AhR antagonists in liver cells. Joiakim et al. (Drug Metab Dispos 31 (2003):1279-82) showed that the Jun N-terminal kinase inhibitor anthra[1,9-cd]pyrazol-6(2H)-one is capable of inhibiting the action of the potent AhR agonist 2,3,7,8-tetrachlorodibenzo-p-dioxin (TCDD) in human breast epithelium cells.

Binding to AhR is also dependent on cell type. Zhang et al. (Environ. Health Perspec. 111 (2003): 1877-1882) have found that for example quercetin prevents the action of AhR in human breast cancer cell line MCF-7, but has no effect on human liver cancer cell line HepG2. A contrary effect has been found for luteolin, which has no effect on MCF-7 cells, but acts as an AhR inhibitor in HepG2 cells. Differences in the ligand affinity of AhR between human cells and rodent cells have also been identified (Ema et al. (1994) J. Biol. Chem. 269: 27337-43; Zhang et al. (2003), Environ. Health Perspec. 111: 1877-1882).

Virtually no compounds are known which act as AhR antagonists in human skin cells. Curcumin does indeed inhibit AhR activation by the tobacco carcinogen benzo[a]pyrene-7R-trans-7,8-dihydrodiol in oral human keratinocyte cancer cells and in ex vivo oral mucosa. However, curcumin activates AhR translocation in the absence of the tobacco carcinogen (Rinaldi et al. (2002) Cancer Res. 62, 5451-5456) and is thus not an AhR antagonist for the purposes of the invention.

It is furthermore known that All-trans-retinoic acid inhibits TCDD-induced AhR activation in normal human keratinocytes, without influencing AhR activity in the absence of TCDD, All-trans-retinoic acid additionally has the considerable disadvantage of enhancing TCDD-induced expression of MMP-1 (Murphy et al. (2004) J. Biol. Chem. 279, 25284-25293) and is photolabile.

The invention accordingly provides Ah receptor modulators (AhR agonists and AhR antagonists) and methods for assessing the effectiveness of a substance to be investigated as an AhR antagonist or AhR agonist. For the purposes of the present invention, an Ah receptor antagonist (AhR antagonist) is a substance which
1. in skin cells, in particular in preferably human melanocytes or keratinocytes,
2. reduces or inhibits AhR-mediated induction of an AhR-inducible gene, preferably of CYP1A1, and, in so doing,
3. in the absence of UVB radiation and/or of an AhR inducer or agonist, preferably a polycyclic aromatic hydrocarbon and particularly preferably benzo[a]pyrene or 3-methylcholanthrene, does not itself trigger induction of an AhR-inducible gene, preferably of CYP3A1, and/or induce translocation of AhR from the cytoplasm into the cell nucleus, and furthermore
4. is preferably photostable.

An Ah receptor agonist (AhR agonist) for the purposes of the present invention is a substance which,
1. in skin cells, in particular in preferably human melanocytes or keratinocytes,
2. induces expression of an AhR-inducible gene, preferably of CYP1A1, and furthermore
3. is preferably photostable.

For the purposes of the present invention, a photostable substance is one which, on irradiation with UVB radiation (40 W/m²) for 2 hours, is no more than 10 mol % converted into one or more other substances. All-trans-retinoic acid, for example, is accordingly not a preferred AhR antagonist, as this substance is photolabile.

For the purposes of the present invention, a gene is deemed to be induced if the concentration of the associated mRNA is significantly (p<0.05, Student's t-test), at least 10%, higher in the presence of the assigned inducer or agonist than in the absence of the inducer or agonist.

An Ah receptor sequence has for example been deposited under NCBI number BC070080.

The invention now provides a method for assessing the effectiveness of an AhR modulator, comprising the steps

i) stressing a cell of an ex vivo or in vitro melanocyte and/or keratinocyte cell culture or of an in vitro skin model with a possible AhR agonist or AhR antagonist,
ii) if the cell was stressed in step i) with a possible AhR antagonist, treating the stressed cell with UVB radiation or an AhR agonist, and
iii) determining induction of an AhR-inducible gene, preferably of CYP1A1.

The assessment method according to the invention now for the first time makes it possible to test substances for their effectiveness as an AhR modulator, i.e. as an Ah receptor agonist or antagonist, without having to carry out this testing intracorporeally on a living animal or living human test subject but instead allowing a representative assessment of the in vivo agonist or antagonist action to be achieved by an ex vivo or in vitro approach. The invention accordingly for the first time opens up the field of Ah receptor agonists and antagonists to systematic investigation involving the use of little material, it being for the first time possible to obtain the results of the assessment rapidly and not, as in the case of intracorporeal treatment in an animal or human test subject, only after a considerable delay, if at all, once further test samples have been taken or once extended observations have been made of treated portions of skin. The method according to the invention accordingly also for the first time makes it possible to carry out a systematic search for substances for reducing or preventing translocation of AhR into a cell nucleus, for reducing or preventing UVB-induced or UVB-inducible gene expression, for reducing or preventing gene expression induced or inducible by polycyclic aromatic hydrocarbons, such as in particular TCDD, and/or for reducing or preventing UVB-induced or UVB-inducible skin damage, in particular skin cancer, skin aging, skin inflammation and sunburn. The method according to the invention likewise makes it possible to carry out systematic testing of potential AhR agonists and reproducibly to determine their action on melanin formation in a cell culture. The assessment method according to the invention furthermore makes it possible to test substances, the toxicity of which had not been verified, or not completely so, before carrying out the assessment method. The assessment method according to the invention accordingly makes further classes of substances which could not previously be assessed on human test subjects accessible to assessment. The method additionally assists in the selection of suitable agonists and antagonists, by permitting standardized and reproducible selection of potential agonists and antagonists with reference to a preselected increase or reduction in induction of the AhR-inducible gene, preferably of CYP1A1.

The cell culture used is preferably a culture consisting of or containing melanocytes and/or keratinocytes. It is likewise preferred to use a cell culture of an ex vivo or in vitro skin model. The entire cell culture is conveniently treated with the potential AhR agonist or antagonist. In preferred embodiments of the invention, the cell culture comprises at least 20 cells, more preferably at least 100 cells and particularly preferably at least 200 cells.

A particularly preferred method for assessing the effectiveness of an AhR modulator comprises the steps:

i) providing four cell groups of an in vitro melanocyte and/or keratinocyte cell culture or of an ex vivo or in vitro skin model containing keratinocytes and optionally additionally melanocytes and further skin cell types,
ii) treating the cell groups in accordance with the following scheme:

| Step | Cell group 1 | Cell group 2 | Cell group 3 | Cell group 4 |
|---|---|---|---|---|
| 1. | Treatment with | No treatment | Treatment with | No treatment |
| | a possible AhR | with a possible | a possible AhR | with a possible |
| | modulator to be | AhR modulator | modulator to be | AhR modulator |
| | investigated, | to be | investigated, | to be |
| | | investigated, | | investigated, |
| 2. | No treatment | Treatment with | Treatment with | No treatment |
| | with a | a preselected | a preselected | with a |
| | preselected | AhR agonist or | AhR agonist or | preselected |
| | AhR agonist or | UVB radiation, | UVB radiation, | AhR agonist or |
| | UVB radiation, | | | UVB radiation, |
| 3. | Determination | Determination | Determination | Determination |
| | of induction of | of induction of | of induction of | of induction of |
| | an AhR- | an AhR- | an AhR- | an AhR- |
| | inducible gene, | inducible gene, | inducible gene, | inducible gene, |
| | preferably of | preferably of | preferably of | preferably of |
| | CYP1A1. | CYP1A1. | CYP1A1. | CYP1A1. |

An AhR modulator is then recognized by the fact that, in cell group 1, the AhR-inducible gene is induced (AhR agonist) or is not induced (AhR antagonist), while in cell group 2 the AhR-inducible gene is induced (positive control). An AhR modulator is likewise recognized by the fact that, in cell group 3, the AhR-inducible gene is induced (AhR antagonist) or is not induced (AhR agonist), while in cell group 4 the AhR-inducible gene is not induced (negative control). The person skilled in the art will understand that the method may also be carried out solely with cell groups 1 and 2 or solely with cell groups 3 and 4.

Unless otherwise stated below, a cell culture of human keratinocytes is used as a keratinocyte cell culture. Preferred keratinocytes are HaCaT cells and NCTC2544 keratinocytes. Preferred melanocytes are murine melanocytes, in particular B16 cells.

A preferred determination method furthermore comprises the steps.

iv) stressing an untreated cell (preferably a preferably murine melanocyte or keratinocyte cell and particularly preferably a B16, NCTC2544 or HaCaT cell) from step i) with a preselected compound as described below, preferably of the formula (II) and particularly preferably of the formula (X),
v) treating the cell stressed in step iv) with UVB radiation and/or an AhR agonist as in step ii),
vi) determining the induction of the AhR-inducible gene, preferably of CYP1A1, determined in step iii), and
vii) comparing the gene induction determined in step iii) and step vi).

The determination method according to the invention may in this manner be particularly straightforwardly standardized and, in an advantageously simple manner, assists in achieving AhR antagonists having a preselected minimum antagonistic action.

In step ii) and optionally in step v), the stressed cell(s) are particularly preferably treated with a polycyclic aromatic hydrocarbon and preferably with TCDD. Such an assessment method according to the invention also makes it possible to analyze whether the assessed AhR antagonist is thus suitable, in addition to detoxification, for preventing or suppressing harmful effects of polycyclic aromatic hydrocarbons and in particular of TCODD. Without the assessment method according to the invention, reliable and systematic detection of such a detoxifying action has previously been virtually impossible, since intentional, controlled exposure of human test subjects to polycyclic aromatic hydrocarbons had to be avoided due to the health risk associated with the use of these substances.

The invention additionally provides a screening method for screening a substance library for AhR agonists and AhR antagonists, comprising the steps:

1) providing a sample of each of the substances in the substance library, and
2) carrying out a method of one of the types previously described for each of the samples provided in 1),
3) selecting those substances, for the samples of which induction of the AhR-inducible gene was reduced by a preselected extent, as AhR antagonists; or selecting those substances, for the samples of which expression of an AhR-inducible gene was increased by a preselected extent, as AhR agonists.

The screening method achieves the advantages associated with the assessment method according to the invention. In particular, it can be carried out as a high throughput method with at least 96 samples, with one or more samples possibly being control samples, in particular positive and/or negative controls. The samples are here preferably arranged together on a support, for example a microtiter plate.

An apparatus according to the invention for carrying out a high throughput screening method according to the invention comprises:
a sample holder for at least 96 samples,
illumination means for stressing the samples with UVB radiation,
means for determining the induction of an AhR-inducible gene in one of the 96 samples, and
evaluation means for indicating induction of the AhR-inducible gene.

The high throughput screening (HTS) apparatus according to the invention advantageously reduces the complexity associated with carrying out a systematic search for AhR agonists and antagonists. Thanks to its preferably automatic mode of operation, it permits standardization of the test conditions, promotes comparability of the evaluation results and so accelerates the search for agonists and antagonists.

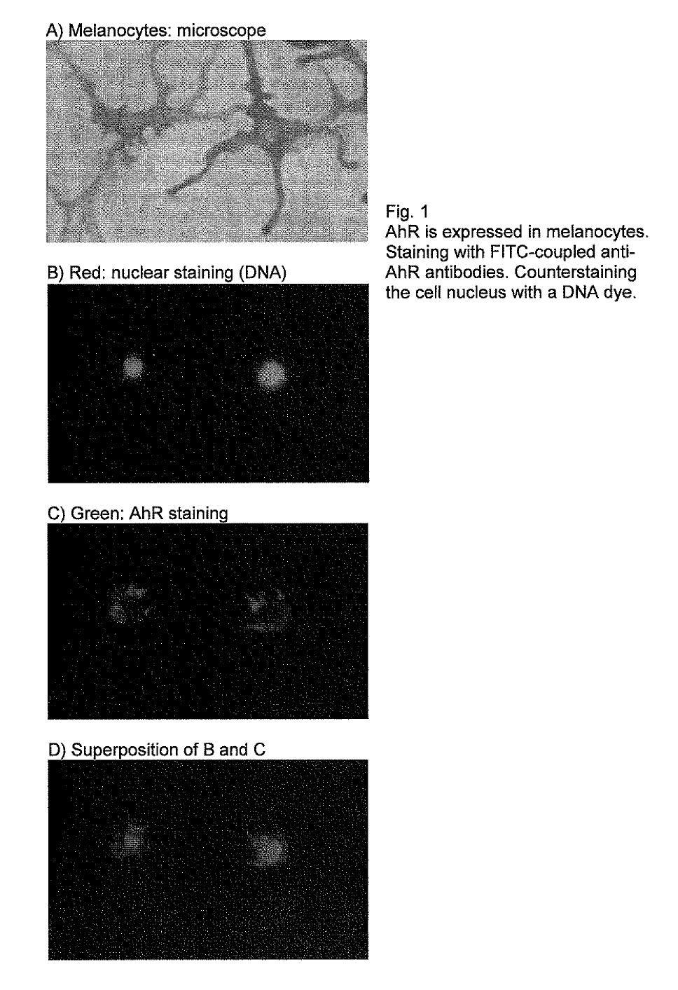

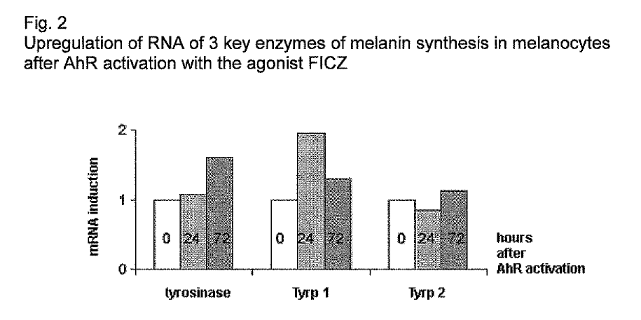

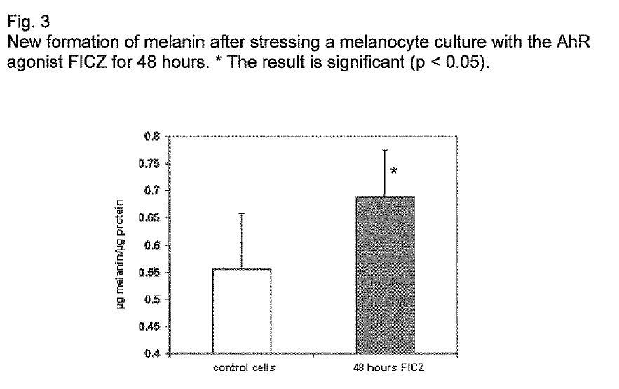

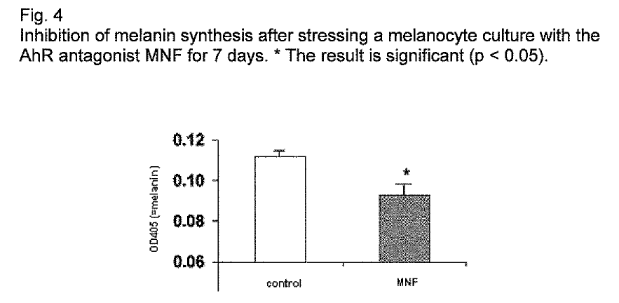

It has now been found that primary skin melanocytes express this receptor (FIG. 1). If has further been found that activation of the receptor with the assistance of an AhR modulator was surprisingly capable of inducing expression of tyrosinase, i.e. the key enzyme in melanin synthesis, by a factor of 2-3 (FIG. 2), and that increased new formation of melanin consequently occurs (FIG. 3). In contrast, inhibitors of the AhR signal pathway have a melanin-reducing action (FIG. 4).

The search for AhR agonists and antagonists is thus in particular directed at the goal of providing agents for protecting or treating animal and preferably human skin. The invention accordingly also provides a method for producing a skin-protection preparation comprising mixing an AhR antagonist, preferably an AhR antagonist found with one of the above-stated methods according to the invention, with a cosmetically and/or pharmaceutically acceptable carrier, such that the concentration of the AhR antagonist in the mixture amounts to at least twice the minimum concentration necessary for reducing induction of an AhR-induced gene, wherein the minimum concentration is determined or determinable using a method according to one of the above-stated methods according to the invention.

The invention accordingly also provides a method for producing a skin-tanning preparation comprising mixing an AhR agonist, preferably an AhR agonist found with one of the above-stated methods according to the invention, with a cosmetically and/or pharmaceutically acceptable carrier, such that the concentration of the AhR agonist in the mixture amounts to at least twice the minimum concentration necessary for increasing expression of an AhR-inducible gene, wherein the minimum concentration is determined or determinable using a method according to one of the above-stated methods according to the invention.

In the skin-protection preparations and skin-tanning agents produced according to the invention, the concentration of AhR antagonists or AhR agonists preferably amounts to no more than 20 times the above-described minimum concentration, particularly preferably no more than 10 times.

The methods according to the invention for assessing the effectiveness of an AhR agonist or antagonist enable the determination of a minimum concentration by carrying out the particular method repeatedly with different concentrations of the particular agonist or antagonist. The methods thus assist in the production of a skin-protection preparation or skin-tanning preparation in that, by making it possible to determine the minimum concentration, they provide an essential parameter for the production of a skin-protection preparation or skin-tanning preparation with elevated accuracy and high predictability with regard to use in humans.

It has now been found that a considerable proportion of AhR antagonists have a skin-lightening action and a considerable proportion of AhR agonists have a skin-tanning action. The invention accordingly also provides a method for assessing the effectiveness of a skin-lightening agent comprising the steps

i) stressing a cell with a possible skin-lightening agent,
ii) determining the extent of melanin formation of the cell treated in step i),
iii) stressing an untreated cell from step i) with a preselected amount of a skin-lightening agent standard, preferably kojic acid or beta-arbutin,
iv) determining the extent of melanin formation of the cell treated in step iii),
v) comparing the skin lightening determined in step ii) and step iv)

Steps iii-v are here optional.

The invention similarly provides a method for assessing the effectiveness of a skin-tanning agent comprising the steps

i) stressing a cell with a possible skin-tanning agent,
ii) determining the extent of melanin formation of the cell treated in step i),
iii) stressing an untreated cell from step i) with a preselected amount of a skin-tanning agent standard, preferably naringin, or caffeine,
iv) determining the extent of melanin formation of the cell treated in step iii),
v) comparing the skin tanning determined in step ii) and step iv).

Steps iii-v are here optional.

The methods accordingly make it possible to achieve the advantages of a method according to the invention for assessing the effectiveness of an AhR antagonist or AhR agonist, in particular reproducibility, the possibility of carrying out a systematic search for skin-lightening agents or skin-tanning agents, high predictability for use in humans and the possibility of carrying out the method as a high throughput method, for example with the assistance of the above-described apparatus according to the invention. The skin-lightening agents investigated in the above method are preferably previously examined for their action as AhR antagonists in a method according to the invention; likewise, the possible skin-tanning agents investigated in the above methods are preferably previously examined for their action as AhR agonists in a method according to the invention.

The cells used in step i) are preferably melanocytes, particularly preferably murine melanocytes. As described above, conveniently at least 20 cells, more preferably at least 100 cells and particularly preferably at least 200 cells are treated.

The invention accordingly likewise provides a method for producing a skin-lightening preparation comprising mixing a skin-lightening agent with a cosmetically and/or pharmaceutically acceptable carrier, such that the concentration of the skin-lightening agent in the mixture amounts to at least twice the minimum concentration necessary for skin-lightening, wherein the minimum concentration is determined with a method of the type just described.

An active ingredient preferred according to the invention for producing a skin-tanning preparation is formylindolo(3,2b)carbazole, and the pharmaceutically acceptable salts and esters thereof. An active ingredient preferred according to the invention for producing a skin-lightening preparation is 3′-methoxy-4′-nitroflavone, and the pharmaceutically acceptable salts and esters thereof.

Some valuable AhR antagonists have been found according to the invention, namely compounds of the formula (II)

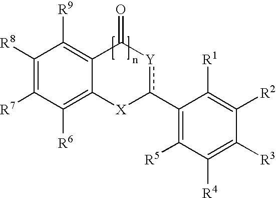

wherein
R¹ to R⁹ mutually independently mean hydrogen, hydroxy, C₁-C₁₂ alkoxy, C₁-C₁₂ alkyl, C₂-C₁₂ alkenyl, and
n=0 and Y=—CH— or —C(CH₃)—, X=O and the dashed line correspondingly means either a double bond or two [hydrogens],
or
n=1, Y=O and X=methylene and the dashed line means two hydrogens.

The compounds of the formula (II) have surprisingly proved to be highly effective Ah receptor antagonists. They are capable of preventing UVB-induced translocation of AhR from the cytoplasm into the cell nucleus in human skin cells. They greatly reduce AhR-mediated induction of AhR-inducible genes in human skin cells, in particular the induction of CYP1A1. They are photostable and, in the absence of an AhR-activating substance, in particular of polycyclic aromatic hydrocarbons such as TCDD, they do not trigger induction of an AhR-inducible gene and also do not induce the translocation of AhR from the cytoplasm into the cell nucleus of human skin cells, in contrast with for example curcumin and All-trans-retinoic acid.

The compounds of the formula (II) are therefore particularly suitable as medicaments, in particular for treating or preventing, in particular UVB-induced, skin irritation, skin damage, skin inflammation, itching, atopical dermatitis, skin aging, skin cancer, and/or for reducing the MMP content of the skin. The compounds are furthermore suitable, as a medicament or in a nonmedicament form, for example as a cosmetic or in cosmetic preparations, for reducing or preventing translocation of AhR into a cell nucleus, for reducing or preventing UVB-induced gene expression, and/or for reducing or preventing gene expression induced by AhR agonists such as polycyclic aromatic hydrocarbons and in particular TCDD. The compounds of the formula (II) are furthermore suitable as sunscreen preparations and in particular as UVB screening agents.

Preferred compounds according to the invention are those in which R¹ to R⁹, mutually independently, mean hydrogen, hydroxy, C₁-C₄ alkoxy (branched or unbranched) or C₂-C₄ alkenyl. Preferably, no more than five and particularly preferably no more than four of the residues R¹ to R⁹ are not hydrogen. If one of the residues is hydroxy or C₁-C₄ alkoxy, then in preferred embodiments the other, thus preferably at most three, residues may be equal to H or C₁-C₄ alkyl (branched or unbranched). Compounds substituted in this manner have proved to be particularly active AhR antagonists.

In particular, the compound of the formula (X) is preferred

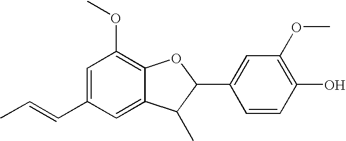

Even when used in very low concentrations, this compound enables strong inhibition of the Ah receptor and, even at low concentrations, prevents or reduces AhR-mediated induction of AhR-inducible genes, in particular CYP1A1. The compound of the formula (X) additionally has a skin-lightening action. Furthermore, in the absence of UVB radiation, the compound of the formula (X) does not itself bring about induction of an AhR-inducible gene, in particular of CYP1A1, and, under these conditions, also do not induce translocation of AhR from the cytoplasm into the cell nucleus. The compound of the formula (X) is therefore particularly suitable for the above-described uses of AhR antagonists, and is particularly preferred as a medicament or constituent of pharmaceutical or nonpharmaceutical and in particular cosmetic preparations.

The invention furthermore provides preparations containing one or more compounds of the formula (II), in particular of the formula (X). The compounds of the formula (II) and in particular the compound of the formula (X) are conveniently present in preparations according to the invention in a sufficient amount (a) for reducing or preventing translocation of AhR into a cell nucleus, (b) for reducing or preventing UVB-induced gene expression, and/or reducing or preventing gene expression induced or inducible by polycyclic aromatic hydrocarbons, preferably TCDD, and/or (d) reducing or preventing UVB-induced or UVB-inducible skin damage, in particular skin cancer, skin aging, skin inflammation and sunburn. The compound of the formula (X) is additionally preferably present in an amount sufficient for skin lightening.

The preparations according to the invention preferably contain the compound(s) of the formula (II), in particular of the formula (X), in a proportion of at least 0.0001 wt. %, relative to the entire preparation. At these concentrations, in particular for the compound of the formula (X), there is already an observable reduction in translocation of the AhR receptor into the cell nucleus of skin cells, and furthermore induction of AhR-inducible genes in particular CYP1A1, for example by polycyclic aromatic hydrocarbons such as TCDD, is already significantly reduced and skin lightening achieved.

The concentration of the compound(s) of the formula (II), in particular of the formula (X), preferably amounts to 0.0005 to 15 wt. %, particularly preferably 0.001 to 10 wt. %, in particular 0.01 to 5 wt. %, in each case relative to the entire composition. At these concentrations, when applied onto the skin, the compounds of the formula (II) have a strong AhR antagonist action by preventing or reducing translocation of AhR into the cell nucleus and in particular reducing or preventing UVB-induced gene expression, especially of CYP1A1.

The preparations may in particular be cosmetic preparations, wherein sunscreen preparations and aftersun preparations are particularly preferred.

The cosmetic or therapeutic formulations according to the invention are produced by conventional, per se known methods in such a manner that the compound(s) of the formula (II), in particular of the formula (X), are incorporated into cosmetic or dermatological formulations, which are of conventional composition and which, in addition to the skin- and hair-lightening action, may also serve to treat, condition and clean the skin or hair.

The formulations according to the invention are preferably present as an emulsion, for example an emulsion of type W/O (water in oil), O/W (oil in water), W/O/W (water in oil in water), O/W/O (oil in water in oil), PIT emulsion, Pickering emulsion, emulsion with a low oil content, micro- or nanoemulsion, as a solution for example in oil (fatty oils or fatty acid esters, in particular C₁-C₃₂ fatty acid C₂-C₃₀ esters) or silicone oil, dispersion, suspension, cream, lotion or milk, depending on production process and constituents, as a gel (including hydrogel hydrodispersion gel, oleogel), spray (for example pump spray or spray with propellant) or also foam or as an impregnation solution for cosmetic tissues, as cleansing agents such as for example soap, synthetic detergent, liquid washing, shower and bath preparation, bath product (capsule, oil, tablet, salt, bath salt, soap, etc.), effervescent preparation, as a skin care product, such as for example emulsion (as described above), ointment, paste, gel (as described above), oil, toner, balsam, serum, powder (for example face powder, body powder), as a mask, as a stick, roll-on, pump, aerosol (foaming, non-foaming or post-foaming), as a deodorant and/or antiperspirant, mouthwash and water pick, as a foot care product (including keratolytic preparation, deodorant), as an insect repellent, as a sunscreen preparation, as an aftersun preparation, as a skin toner, as a shaving preparation, aftershave balm, pre- and aftershave lotion, as a depilatory product, as a hair care product such as for example shampoo (including 2-in-1 shampoo, antidandruff shampoo, baby shampoo, shampoo for dry scalp, shampoo concentrate), conditioner, hair tonic, hair lotion, hair rinse, hair cream, pomade, permanent wave and setting lotion, hair strengthener (spray), styling aid (for example gel or wax), hair smoothing product (defrizzing agent, relaxer), as a blonding product, hair dyes such as for example temporary, direct hair dyes, semipermanent hair dyes, permanent hair dyes, hair toning product, hair lightening agent, hair conditioner, hair mousse, eyecare preparations, make-up, make-up remover or baby products.

It is also advantageous to present the compound(s) of the formula (II), in particular of the formula (X), in encapsulated form, for example in gelatin, wax materials, liposomes or cellulose capsules.

The formulations according to the invention are particularly preferably present in the form of an emulsion, in particular in the form of a emulsion of the W/O, O/W, W/O/W, O/W/O type, PIT emulsion, Pickering emulsion, emulsion with a low oil content, micro- or nanoemulsion, a gel (including hydrogel, hydrodispersion gel, so oleogelt), a solution for example in oil (fatty oils or fatty acid esters, in particular C₆-C₃₂ fatty acid C₂-C₃₀ esters)) or silicone oil, or a spray (for example pump spray, or spray with propellant).

The (in particular topical) cosmetic or therapeutic formulations according to the invention may preferably contain cosmetic and/or dermatological auxiliary substances and additives, as are conventionally used in such formulations, for example cooling active ingredients, sunscreen active ingredients (in particular UV filters, and/or UV-filtering pigments), dyes, pigments which have a coloring action, antioxidants, preservatives, antiirritants, softening, moistening (moisture-donating) and/or moisture-retaining substances (moisture-retaining regulators, for example glycerol or urea), osmolytes, antimicrobial active ingredients (for example antibacterial active ingredients, bactericides, fungicides), virucides, deodorants (for example antiperspirant agents), surface-active substances (surfactants), emulsifiers, insect repellents (for example DEET, IR 3225, Dragorepel), plant extracts, antiinflammatory active ingredients (antiinflammatory agents), substances which accelerate wound healing (for example chitin or chitosan and the derivatives thereof, gel-forming agents, film-forming substances (film formers, for example polyvinylpyrrolidone or chitosan or the derivatives thereof, fixatives, skin-smoothing active ingredients, wrinkle-reducing active ingredients such as beta-glucan from oats, or blackberry leaf extract or soy extract, vitamins (for example vitamin C and derivatives, tocopherols and derivatives, vitamin A and derivatives), 2-hydroxycarboxylic acids (for example citric acid, malic acid, L-, D- or DL-lactic acid), skin dyes (for example walnut extracts, or dihydroxyacetone), skin-conditioning and -repairing agents (for example cholesterol, ceramides, pseudoceramides, creatine and creatine esters), skin-soothing agents, moisturizing agents, optical brighteners, slip agents, lustrants, fats, oils, saturated fatty acids and the salts thereof mono- or polyunsaturated fatty acids and the salts thereof, alpha-hydroxy acids, polyhydroxyfatty acids or the derivatives thereof (for example linoleic acid, alpha-linolenic acid, gamma-linolenic acid or arachidonic acid and the respective natural or synthetic esters thereof), phospholipids, waxes or other conventional constituents of a cosmetic or dermatological formulation such as alcohols, alkanediols, polyols, polymers, electrolytes, organic solvents, silicones, silicone derivatives or chelating agent (for example ethylenediaminetetraacetic acid and derivatives), antidandruff active ingredients (antidandruff agents, for example climbazole, ketoconazole, piroctone olamine, zinc pyrithione), hair care products, hair styling products, hair smoothing products, depilatory products, perfumes, essential oils, foaming agents, foam stabilizers, foam boosters, substances for preventing foaming, thickeners, binders, plant parts (for example fibers) and plant extracts (for example arnica, aloe, beard lichen, ivy, stinging nettle, ginseng, henna, chamomile, marigold, rosemary, sage, horsetail or thyme), animal extracts such as for example royal jelly, propolis, proteins, protein hydrolysates, yeast extracts, hop and wheat extracts, peptides or thymus extracts, abrasives (abrasive media), buffers, enzymes.

Ingredients (auxiliary substances and additives) with which the compound(s) of the formula (II), in particular of the formula (X), may be combined are particularly preferably:

abrasives, antidandruff agents, antiinflammatory agents, antioxidants, antiperspirant agents, binders, buffers, chelating agents, depilatory products, surface-active substances, emulsifiers, enzymes, essential oils, plant extracts, fibers, film formers, fixatives, foaming agents, foam stabilizers, substances for preventing foaming, foam boosters, gel-forming agents, hair care products, hair styling products, hair smoothing products, active ingredients for skin- and hair-lightening, moisture-donating agents, moisture-retaining substances, insect repellents, optical brighteners, slip agents, lustrants, polymers, proteins, moisturizing agents, skin-soothing agents, skin-smoothing active ingredients, wrinkle-reducing active ingredients, sunscreen active ingredients, vitamins, oils, waxes, fats, phospholipids, saturated fatty acids and the salts thereof, mono- or polyunsaturated fatty acids and the salts thereof alpha-hydroxy acids, polyhydroxyfatty acids, polyols, alkanediols, silicones or silicone derivatives.

Auxiliary substances and additives may be present in amounts of 5 to 99 wt. %, preferably of 10 to 80 wt. %, relative to the total weight of the formulation. Straightforward trials can be carried out by a person skilled in the art to determine the particular amounts of cosmetic or dermatological auxiliary substances and additives and perfume to be used depending on the nature of the particular product.

The formulations may furthermore comprise water in an amount of up to 99.99 wt. %, preferably of 5 to 80 wt. %, relative to the total weight of the formulation.

Cosmetic or therapeutic formulations according to the invention are preferably those formulations,

which are selected from the group consisting of emulsion, solution, dispersion, suspension, cream, lotion, milk, gel, spray, foam, impregnation solution for cosmetic tissues, cleaning agents, soap, synthetic detergent, washing preparation, shower preparation, bath preparation, bath product, effervescent preparation, skin care product, ointment, paste, oil, toner, balsam, serum, powder, mask, stick, roll-on, pump, aerosol, deodorant, antiperspirant, mouthwash, water pick, foot care product, insect repellent, sunscreen preparation, self-tanning preparation, aftersun preparation, skin toner, shaving preparation, aftershave balm, preshave lotion, aftershave lotion, depilatory product, hair care products, shampoo, conditioner, hair tonic, hair lotion, hair rinse, hair cream, pomade, permanent wave preparation, setting lotion, hair strengthener, styling aid, hair smoothing products, blonding product, hair dye, hair toning product, hair lightening agent, hair conditioner, hair mousse, eye care cream, make-up, make-up remover and baby products, and/or

which, in addition to compound(s) of the formula (II), in particular of the formula (X), contain one or more auxiliary substances and additives selected from the group consisting of

abrasives, antidandruff agents, antiinflammatory agents, antioxidants, antiperspirant agents, binders, buffers, chelating agents, depilatory products, surface-active substances, emulsifiers, enzymes, essential oils, plant extracts, fibers, film formers, fixatives, foaming agents, foam stabilizers, substances for preventing foaming, foam boosters, gel-forming agents, hair care products, hair styling products, hair smoothing products, active ingredients for skin- and hair-lightening, moisture-donating agents, moisture-retaining substances, insect repellents, optical brighteners, slip agents, lustrants, polymers, proteins, moisturizing agents, skin-soothing agents, skin-smoothing active ingredients, wrinkle-reducing active ingredients, sunscreen active ingredients, vitamins, oils, waxes, fats, phospholipids, saturated fatty acids and the salts thereof, mono- or polyunsaturated fatty acids and the salts thereof, alpha-hydroxy acids, polyhydroxyfatty acids, polyols, alkanediols, silicones and silicone derivatives,

and/or
which are intended for application onto the hair and/or the skin.

Formulations containing the compound(s) of the formula (II), in particular of the formula (X), are generally applied in a sufficient amount onto the skin and/or hair in the manner conventional for cosmetics and skin preparations. Such cosmetic, dermatological and/or therapeutic formulations according to the invention which additionally comprise one or more sunscreen filters (UV absorbers, UV filters,) offer particular advantages.

In rare cases, discoloration and/or instabilities may occur in formulations containing compounds of the formula (II) according to the invention or to be used according to the invention, in particular if they are present in aqueous-alcoholic or pure alcoholic solutions. It has surprisingly now been found that UV filters are capable of improving the stability of the compounds of the formula (II) in formulations according to the invention. In particular, UV filters are capable of preventing or retarding discoloration of the compounds of the formula (II) brought about by sunlight or other light. Both factors are of significance in particular in cosmetic formulations. According to the invention, UV filters are therefore used for stabilizing the compounds of the formula (II), in particular by one or more UV filters being used in a formulation according to the invention in an amount sufficient for stabilizing the compounds of the formula (II), preferably by using the (preferred) UV filters stated further below. In this connection, a further aspect of the invention relates to the cosmetic or therapeutic use of one or more compounds of the formula (II) for lightening skin and/or hair in the presence of an amount of one or more UV filters which stabilizes the compound or compounds of the formula (II), in particular of the formula (X), wherein all the details regarding selection of the substituents stated further above naturally also apply in this respect. For the purposes of stabilization, the total amount of UV filters is preferably in the range from 0.1 to 2 wt. %, in particular 0.2 to 1 wt. %, relative to the total weight of the formulation.

The ratio of the total proportion by weight of UV filters to the total proportion by weight of the compounds of the formula (II) according to the invention or to be used according to the invention is preferably in the range from 100:1 to 1:100, particularly preferably in the range from 10:1 to 1:10, very particularly preferably in the range from 5:1 to 1:5.

Formulations according to the invention containing one or more UV filters (sunscreen filters, UV absorbers) preferably comprise a total proportion of UV filters in the range from 0.1 to 30 wt. %, particularly preferably in the range from 0.2 to 20 wt. %, very particularly preferably in the range from 0.5 to 15 wt. %, relative to the total weight of the formulation. The formulations according to the invention particularly preferably contain one or more UVB filters, in particular of types stated below. It has been found that the substances of the formula (II) and in particular of the formulae (X) according to the invention interact advantageously with UVB filters to prevent UVB-induced skin damage, skin changes and skin cancer.

The compounds of the formula (II) and in particular of the formula (X) according to the invention or to be used according to the invention are particularly preferably combined with water-soluble UV filters, in a preferred development with phenylene-bis-benzimidazyl-tetrasulfonic acid disodium salt (Neo Heliopan®AP) and/or 2-phenylbenzimidazolesulfonic acid (Neo Heliopan®Hydro).

In a further preferred development, a formulation according to the invention contains a total amount of sunscreen active ingredients, i.e. in particular UV filters and/or inorganic pigments (UV-filtering pigments), such that the formulation according to the invention exhibits a sun protection factor of greater than or equal to 2 (preferably greater than or equal to 5). Such formulations according to the invention are particularly suitable for protecting skin and hair.

Formulations according to the invention additionally comprising one or more sunscreen filters (UV filters, UV absorbers) may here be present in different forms, as they are for example conventionally used for sunscreen formulations. They may, for example, be present in the form of an emulsion of the oil-in-water (O/W) type, a gel, a hydrodispersion, or also an aerosol.

The formulations according to the invention advantageously contain at least one UVA filter and/or at least one UVB filter and/or a broadband filter and/or at least one inorganic pigment. Formulations according to the invention preferably contain at least one UVB filter or a broadband filter, further particularly preferably at least one UVA filter and at least one UVB filter.

Suitable UV filters, are for example organic UV absorbers from the class of 4-aminobenzoic acid and derivatives, salicylic acid derivatives, benzophenone derivatives, dibenzoylmethane derivatives, diphenyl acrylates, 3-imidazol-4-yl-acrylic acid and the esters thereof, benzofuran derivatives, benzylidene malonate derivatives, polymeric UV absorbers containing one or more organosilicon residues, cinnamic acid derivatives, camphor derivatives, trianilino-s-triazine derivatives, 2-hydroxyphenylbenzotriazole derivatives, phenylbenzimidazole-sulfonic acid derivatives and the salts thereof, anthranilic acid menthyl esters, benzotriazole derivatives, indole derivatives.

The UV filters stated below, which may be used for the purposes of the present invention, are preferred but are, of course, not limiting.

Advantageous UV filters are

UVB filters such as for example:
p-aminobenzoic acid
p-aminobenzoic acid ethyl ester (25 mol), ethoxylated (INCI name: PEG-25 PABA)
p-dimethylaminobenzoic acid 2-ethylhexyl ester
p-aminobenzoic acid ethyl ester (2 mol), N-propoxylated
p-aminobenzoic acid glycerol ester
salicylic acid homomethyl ester (Homosalate) (Neo Heliopan®HMS)
salicylic acid 2-ethylhexyl ester (Neo Heliopan®OS)
triethanolamine salicylate
4-isopropylbenzyl salicylate
anthranilic acid menthyl ester (Neo Heliopan®MA)
diisopropylcinnamic acid ethyl ester
p-methoxycinnamic acid 2-ethylhexyl ester (Neo Heliopan®AV)
diisopropylcinnamic acid methyl ester
p-methoxycinnamic acid isoamyl ester (Neo Heliopan®E 1000)
p-methoxycinnamic acid diethanolamine salt
p-methoxycinnamic acid isopropyl ester
2-phenylbenzimidazolsulfonic acid and salts (Neo Heliopan®Hydro)
3-(4′-trimethylammonium)-benzylidene-bornan-2-one methyl sulfate
beta-imidazole-4(5)-acrylic acid (urocaninic acid)
3-(4′-sulfo)benzylidene-bornan-2-one and salts
3-(4′-methylbenzylidene)-D,L-camphor (Neo Heliopan®MBC)
3-benzylidene-D,L-camphor
N-[(2 and 4)-[2-(oxoborn-3-ylidene)methyl]benzyl]acrylamide polymer
4,4′-[(6-[4-(1,1-dimethyl)-aminocarbonyl)-phenylamino]-1,3,5-triazine-2,4-diyl)diimino]-bis-(benzoic acid 2-ethylhexyl ester) (Uvasorb®HEB)
benzylidene malonate/polysiloxane (Parsol®SLX)
glyceryl ethyl hexanoate dimethoxycinnamate
dipropylene glycol salicylate
tris(2-ethylhexyl)-4,4′,4″-(1,3,5-triazine-2,4,6-triyltriimino)tribenzoate (=2,4,6-trianilino-(p-carbo-2′-ethylhexyl-1′-oxy)-1,3,5-triazine) (Uvinul®T150)

Broadband filters such as for example.
2-ethylhexyl-2-cyano-3,3-diphenyl acrylate (Neo Heliopan®303)
ethyl-2-cyano-3,3′-diphenyl acrylate
2-hydroxy-4-methoxybenzophenone (Neo Heliopan®BB)
2-hydroxy-4-methoxybenzophenone 5-sulfonic acid
dihydroxy-4-methoxybenzophenone
2,4-dihydroxybenzophenone
tetrahydroxybenzophenone
2,2′-dihydroxy-4,4′-dimethoxybenzophenone
2-hydroxy-4-n-octoxybenzophenone
2-hydroxy-4-methoxy-4′-methylbenzophenone
sodium hydroxymethoxybenzophenone sulfonate
disodium 2,2′-dihydroxy-4,4′-dimethoxy-5,5′-disulfobenzophenone
phenol, 2-(2H-benzotriazol-2-yl)-4-methyl-6-(2-methyl-3(1,3,3,3-tetramethyl-1-(trimethylsilyl)-oxy)-disiloxyanyl)-propyl), (Mexoryl®XL)
2,2′-methylene-bis-(6-(2H-benzotriazol-2-yl)-4-1,1,3,3-tetramethylbutyl)-phenol), (Tinosorb®M)
2,4-bis-[4-(2-ethythexyl)-2-hydroxyphenyl]-1,3,5-triazine
2,4-bis-[{(4-(2-ethyl-hexyloxy)-2-hydroxy}-phenyl]-6-(4-methoxyphenyl)-1,3,5-triazine, (Tinosorb®S)
2,4-bis-[{(4-(3-sulfonato)-2-hydroxy-propyloxy)-2-hydroxy}-phenyl]-6-(4-methoxyphenyl)-1,3,5-triazine sodium salt
2,4-bis-[{(3-(2-propyloxy)-2-hydroxy-propyloxy)-2-hydroxy}-phenyl]-6-(4-methoxyphenyl)-1,3,5-triazine
2,4-bis-[{4-(2-ethyl-hexyloxy)-2-hydroxy}-phenyl]-6-[4-(2-methoxyethyl-carbonyl)-phenylamino]-1,3,5-triazine
2,4-bis-[{4-(3-(2-propyloxy)-2-hydroxy-propyloxy)-2-hydroxy}-phenyl]-6-[4-(2-ethylcarboxyl)-phenylamino]-1,3,5-triazine
2,4-bis-[{4-(2-ethyl-hexyloxy)-2-hydroxy}-phenyl]-6-(1-methyl-pyrrol-2-yl)-1,3,5-triazine
2,4-bis-[{4-tris-(trimethylsiloxy-silylpropyloxy)-2-hydroxy}-phenyl]-6-(4-methoxyphenyl)-1,3,5-triazine
2,4-bis-[{4-(2″-methylpropenyloxy)-2-hydroxy}-phenyl]-6-(4-methoxyphenyl)-1,3,5-triazine
2,4-bis-[{4-(1′,1′,1′,3′,5′,5′,5′-heptamethylsiloxy-2″-methyl-propyloxy)-2-hydroxyl}-phenyl]-6-(4-methoxyphenyl)-1,3,5-triazine

UVA filters such as for example
4-isopropyldibenzoylmethane
terephthalylidene-dibornanesuifonic acid and salts (Mexoryl®SX)
4-t-butyl-4′-methoxy-dibenzoylmethane (Avobenzon)/(Neo Heliopan®357)
phenylene-bis-benzimidazyl-tetrasulfonic acid disodium salt (Neo Heliopan®AP)
2,2′-(1,4-phenylene)-bis-(1H-benzimidazole-4,6-disulfonic acid), monosodium salt
2-(4-diethylamino-2-hydroxybenzoyl)-benzoic acid hexyl ester (Uvinul®A Plus)
indanylidene compounds according to DE 100 55 940 (=WO 02/38537)

UV absorbers particularly suitable for combining are
p-aminobenzoic acid
3-(4′-trimethylammonium)-benzylidene-bornan-2-one methyl sulfate
salicylic acid homomethyl ester (Neo Heliopan®HMS)
2-hydroxy-4-methoxy-benzophenone (Neo Heliopan®BB)
2-phenylbenzimidazolsulfonic acid (Neo Heliopan®Hydro)
terephthalylidene-dibornanesulfonic acid and salts (Mexoryl®SX)
4-tert.-butyl-4′-methoxydibenzoylmethane (Neo Heliopan®357)
3-(4′-sulfo)benzylidene-bornan-2-one and salts
2-ethylhexyl-2-cyano-3,3-diphenyl acrylate (Neo Heliopan®303)
N-[(2 and 4)-[2-(oxoborn-3-ylidene)methyl]benzyl]acrylamide polymer
p-methoxycinnamic acid 2-ethylhexyl ester (Neo Heliopan®AV)
p-aminobenzoic acid ethyl ester (25 mol), ethoxylated (INCI name: PEG-25 PABA)
p-methoxycinnamic acid isoamyl ester (Neo Heliopan®E1000)
2,4,6-trianilino-(p-carbo-2′-ethylhexyl-1′-oxy)-1,3,5-triazine (Uvinul®T150)
phenol, 2-(2H-benzotriazol-2-yl)-4-methyl-6-(2-methyl-3(1,3,3,3-tetra-methyl 1-(trimethylsilyl)-oxy)-disiloxyanyl)-propyl), (Mexoryl®XL)
4,4′-[(6-[4-(1,1-dimethyl)-aminocarbonyl)-phenylamino]-1,3,5-triazine-2,4-diyl)diimino]-bis-(benzoic acid 2-ethylhexyl ester) (Uvasorb HEB)
3-(4′-methylbenzylidene)-D, L-camphor (Neo Heliopan®MBC)
3-benzylidenecamphor
salicylic acid 2-ethylhexyl ester (Neo Heliopan®OS)
4-dimethylaminobenzoic acid 2-ethylhexyl ester (Padimate O)
hydroxy-4-methoxy-benzophenone 5-sulfonic acid and Na salt
2,2′-methylene-bis-(6-(2H-benzotriazol-2-yl)-4-1,1,3,3-tetramethylbutyl)-phenol), (Tinosorb®M)
phenylene-bis-benzimidazyl-tetrasulfonic acid disodium salt (Neo Heliopan®AP)
2,4-bis-[{(4-(2-ethyl-hexyloxy)-2-hydroxy}-phenyl]-6-(4-methoxyphenyl)-1,3,5-triazine, (Tinosorb®S)
benzylidene malonate/polysiloxane (Parsol®SLX)
menthylanthranilate (Neo Heliopan®MA)
2-(4-diethylamino-2-hydroxybenzoyl)-benzoic acid hexyl ester (Uvinul® A Plus)
indanylidene compounds according to DE 100 55 940 (=WO 02/38537)

Particulate UV filters or inorganic pigments may furthermore be used, which may optionally be hydrophobized, such as the oxides of titanium (TiO₂), zinc (ZnO), iron (Fe₂O₃), zirconium (ZrO₂), silicon (SiO₂), manganese (for example MnO), aluminum (Al₂O₃), cerium (for example Ce₂O₃) and/or mixtures thereof.

Formulations according to the invention, in particular dermatological formulations, may furthermore advantageously contain dyes and/or coloring pigments, in particular if they are to be used in the field of decorative cosmetics. The dyes and pigments may be selected from the corresponding positive list of the German Cosmetics Ordinance or the EC list of cosmetic colorants. In most cases, these are identical with the dyes authorized for foodstuffs. Advantageous coloring pigments are for example titanium dioxide, mica, iron oxide (for example Fe₂O₃ Fe₃O₄, FeO(OH)) and/or tin oxide. Advantageous dyes are for example carmine, Prussian blue, chromium oxide green, ultramarine blue and/or manganese violet.

Individual cooling active ingredients preferred for use for the purposes of the present invention are listed below. A person skilled in the art will be able to supplement this list with many further cooling active ingredients; the listed cooling active ingredients may also be used in combination with one another: L-menthol, D-menthol, racemic menthol, menthone glycerol acetal (trade name: Frescolat®MGA), menthyl lactate (trade name: Frescolat®ML, the menthyl lactate preferably comprising L-menthyl lactate, in particular L-menthyl L-lactate), substituted menthyl 3-carboxamides (for example menthyl 3-carboxylic acid N-ethylamide), 2-isopropyl-N-2,3-trimethylbutanamide, substituted cyclohexane carboxamides, 3-menthoxypropane-1,2-diol, 2-hydroxyethylmenthyl carbonate, 2-hydroxypropylmenthyl carbonate, N-acetyl glycine menthyl ester, isopulegol, menthylhydroxycarboxylic acid ester (for example menthyl 3-hydroxybutyrate), monomenthyl succinate, 2-mercaptocyclodecanone, menthyl 2-pyrrolidin-5-one carboxylate, 2,3-dihydroxy-p-menthane, 3,3,5-trimethylcyclohexanone glycerol ketal, 3-menthyl 3,6-di- and -trioxaalkanoates, 3-menthyl methoxyacetate, icilin.

Preferred cooling active ingredients are: L-menthol, D-menthol, racemic menthol, menthone glycerol acetal (trade name: Frescolat®MGA), menthyl lactate (preferably L-menthyl lactate, in particular L-menthyl L-lactate, trade name: Frescolat®ML), substituted menthyl 3-carboxamides (for example menthyl 3-carboxylic acid N-ethylamide), 2-isopropyl-N-2,3-trimethylbutanamide, substituted cyclohexane carboxamides, 3-menthoxypropane-1,2-diol, 2-hydroxyethylmenthyl carbonate, 2-hydroxypropylmenthyl carbonate, isopulegol.

Particularly preferred cooling active ingredients are: L-menthol, racemic menthol, menthone glycerol acetal (trade name: Frescolat®MGA), menthyl lactate (preferably L-menthyl lactate, in particular L-menthyl L-lactate, trade name: Frescolat®ML), 3-menthoxypropane-1,2-diol, 2-hydroxyethylmenthyl carbonate, 2-hydroxypropylmenthyl carbonate.

Very particularly preferred cooling active ingredients are: L-menthol, menthone glycerol acetal (trade name: Frescolat®MGA), menthyl lactate (preferably L-menthyl lactate, in particular L-menthyl L-lactate, trade name. Frescolat®ML).

The usage concentration of the cooling active ingredients to be used lies, depending on the substance, preferably in the concentration range of from 0.01 to 20 wt. % and particularly preferably in the concentration range of from 0.1 to 5 wt. %, relative to the total mass of the finished (ready-to-use), preferably topical, cosmetic or therapeutic (pharmaceutical) formulation.

The formulations according to the invention may preferably contain further active ingredients for skin- and hair-lightening which are suitable for cosmetic (for example dermatological) and/or therapeutic applications. Advantageous skin-lightening active ingredients are in this respect kojic acid (5-hydroxy-2-hydroxymethyl-4-pyranone), kojic acid derivatives such as for example kojic acid palmitate, arbutin, ascorbic acid, ascorbic acid derivatives, hydroquinone, hydroquinone derivatives, resorcinol, sulfur-containing molecules such as for example glutathione, or cysteine, alpha-hydroxy acids, (for example citric acid, lactic acid, malic acid) and the derivatives thereof, N-acetyl tyrosine and derivatives, undecenoyl phenylalanine, gluconic acid, 4-alkylresorcinols, 4-(1-phenylethyl)1,3-dihydroxybenzene, chromone derivatives such as aloesin, flavonoids, thymol derivatives, 1-aminoethylphosphinic acid, thiourea derivatives, ellagic acid, nicotinamide (niacinamide), zinc salts such as for example zinc chloride or gluconate, thujaplicin and derivatives, triterpenes such as maslinic acid, sterols such as ergosterol, benzofuranones such as senkyunolide, vinyl and ethyl guaiacol, dione acids such as octodecene dione acid and azelaic acid, inhibitors of nitrogen oxide synthesis such as for example L-nitroarginine and the derivatives thereof, 2,7-dinitroindazole or thiocitrulline, metal chelators (for example alpha-hydroxyfatty acids, palmitic acid, phytic acid, lactoferrin, humic acid, bile acid, bile extracts, bilirubin, biliverdin, EDTA, EGTA and the derivatives thereof, retinoids, soy milk and extract, serine protease inhibitors or lipoic acid or other synthetic or natural active ingredients for lightening skin and hair, wherein the latter may also be used in the form of an extract from plants, such as for example bearberry extract, rice extract, papaya extract, liquorice root extract or concentrated constituents thereof such as glabridin, or licochalcone A, Artocarpus extract, extract of Rumex and Ramulus species, extracts from pine species (Pinus) and extracts from Vitis species or stilbene derivatives concentrated therefrom, extract of saxifrage, mulberry, skullcap and/or grapes.

The further active ingredients for skin- and hair-lightening (one or more compounds) stated above by way of example are then preferably present in the formulations according to the invention in an amount of 0.005 to 30 wt. %, preferably of 0.01 to 20 wt. %, particularly preferably of 0.01 to 5 wt. %, relative to the total weight of the formulation.

In formulations according to the invention (in particular if application in the facial area is intended), it may be advantageous to select one or more substances from the following group as dye: 2,4-dihydroxyazobenzene, 1-(2′-chloro-4′-nitro-1′-phenylazo)-2-hydroxynaphthalene, Ceres Red, 2-(4-sulfo-1-naphthylazo)-1-naphthol-4-sulfonic acid, calcium salt of 2-hydroxy-1,2′-azonaphthalene-1′-sulfonic acid, calcium and barium salts of 1-(2-sulfo-4-methyl-1-phenylazo)-2-naphthylcarboxylic acid, calcium salt of 1-(2-sulfo-1-naphthylazo)-2-hydroxynaphthalene-3-carboxylic acid, aluminum salt of 1-(4-sulfo-1-phenylazo)-2-naphthol-6-sulfonic acid, aluminum salt of 1-(4-sulfo-1-naphthylazo)-2-naphthol-3,6-disulfonic acid, 1-(4-sulfo-1-naphthylazo)-2-naphthol-6,8-disulfonic acid, aluminum salt of 4-(4-sulfo-1-phenylazo)-1-(4-sulfophenyl)-5-hydroxy-pyrazolone-3-carboxylic acid, aluminum and zirconium salts of 4,5-dibromofluorescein, aluminum and zirconium salts of 2,4,5,7-tetrabromofluorescein, 3′,4′,5′,6′-tetrachloro-2,4,5,7-tetrabromfluorescein and the aluminum salt thereof, aluminum salt of 2,4,5,7-tetraiodofluorescein, aluminum salt of quinophthalone-disulfonic acid, aluminum salt of indigodisulfonic acid, red and black iron oxide (Colour Index no. (CIN): 77491 (red) and 77499 (black)), iron oxide hydrate (CIN: 77492), manganese ammonium diphosphate and titanium dioxide.

Oil-soluble natural dyes, such as for example capsicum extracts, β-carotene or cochineal.

Dermatological formulations with a content of pearlescent pigments are furthermore advantageous for the purposes of the present invention. The pearlescent pigment types listed below are in particular preferred:

1. Natural pearlescent pigments, such as for example.
“pearl essence” (guanine/hypoxanthine mixed crystals prepared from fish scales) and
“mother of pearl” (ground mussel shells)

2. Monocrystalline pearlescent pigments such as for example bismuth oxychloride (BiOCl)

3. Coated substrate pigments: for example mica/metal oxide

Pearlescent pigments are for example based on pulverulent pigments or castor oil dispersions of bismuth oxychloride and/or titanium dioxide as well as bismuth oxychloride and/or titanium dioxide on mica. The lustrous pigment listed under CIN 77163 is, for example, in particular advantageous.

The list of the stated pearlescent pigments should, of course, not be regarded as limiting. Pearlescent pigments which are advantageous for the purposes of the present invention are obtainable by many per se known methods. For example, substrates other than mica may be coated with further metal oxides, such as for example silica and the like. For example, SiO₂ particles coated with TiO₂ and Fe₂O₃ (“Ronaspheres”), which are distributed by Merck and are particularly suitable for the optical reduction of fine wrinkles, are advantageous.

It may furthermore be advantageous to completely dispense with a substrate such as mica. Iron pearlescent pigments which are produced without using mica are particularly preferred. Such pigments are, for example, obtainable from BASF under the trade name Sicopearl Copper 1000.

Special effect pigments, which are obtainable in various colors (yellow, red, green blue) from Flora Tech under the commercial name Metasomes Standard/Glitter are furthermore particularly advantageous. The glitter particles are here present in mixtures with various auxiliary substances and dyes (such as for example the dyes having the CINs 19140, 77007, 77289, 77491).

The dyes and pigments may be present both individually and as a mixture and be coated with one another, different color effects generally being obtained by different coating thicknesses. The total amount of dyes and color-imparting pigments is advantageously selected from the range of for example 0.1 wt. % to 30 wt. %, preferably from 0.5 to 15 wt. %, in particular from 1.0 to 10 wt. %, in each case relative to the total weight of the (cosmetic) formulations.

The formulations according to the invention may also contain (additional) antioxidants or preservatives. Antioxidants or preservatives which may be used are any antioxidants which are suitable for or conventional in cosmetic (for example dermatological) and/or therapeutic applications.

Antioxidants for the purposes of the invention are any substances which reduce the amount of free radicals in cells and tissues. Antioxidants are advantageously selected from the group consisting of amino acids (for example glycine, histidine, tyrosine, tryptophan) and the derivatives thereof, imidazoles (for example urocahinic acid) and the derivatives thereof peptides such as D,L-carnosine, D-carnosine, L-carnosine and the derivatives thereof (for example Anserin), carotenoids, carotenes (for example alpha-carotene, beta-carotene, lycopene) and the derivatives thereof, lipoic acid and the derivatives thereof (for example dihydrolipoic acid), aurothioglucose, propylthiouracil and other thiols (for example thioredoxin, glutathione, cysteine, cystine, cystamine and the glycosyl, N-acetyl, methyl, ethyl, propyl, amyl, butyl and lauryl, palmitoyl, oleyl, gamma-linoleyl, cholesteryl, glyceryl, and oligoglyceryl esters) and the salts thereof, dilauryl thiodipropionate, distearyl thiodipropionate, thiodipropionic acid and the derivatives thereof (esters, ethers, peptides, lipids, nucleotides, nucleosides and salts) and sulfoximine compounds (for example buthionine sulfoximines, homocysteine sulfoximine, buthionine sulfones, penta-, hexa-, heptathionine sulfoximine) in very small compatible rates of addition (for example pmol to pmol/kg), also (metal) chelating agents (for example alpha-hydroxyfatty acids, palmitic acid, phytic acid, lactoferrin, alpha-hydroxy acids, (for example citric acid, lactic acid, malic acid), humic acid, bile acid, bile extracts, tannins, bilirubin, biliverdin, EDTA, EGTA and the derivatives thereof, unsaturated fatty acids and the derivatives thereof (for example gamma-linolenic acid, linoleic acid, oleic acid), folic acid and the derivatives thereof, ubiquinone and ubiquinol and the derivatives thereof, vitamin C and derivatives (for example ascorbyl palmitate, Mg ascorbyl phosphate, ascorbyl acetate, ascorbyl glucoside), tocopherols and derivatives (for example vitamin E acetate), vitamin A and derivatives (vitamin A palmitate) as well as coniferyl benzoate of benzoic resin, rutinic acid and the derivatives thereof, flavonoids and the glycosylated precursors thereof, in particular quercetin, and the derivatives thereof for example alpha-glucosylrutin, rosmarinic acid, carnosol, carnosolic acid, resveratrol, caffeic acid and the derivatives thereof, sinapic acid and the derivatives thereof, ferulic acid and the derivatives thereof, curcuminoids, chlorogenic acid and the derivatives thereof, retinoids, ursolic acid, levulinic acid, butylhydroxytoluene, butylhydroxyanisole, nordihydroguaiac resin acid, nordihydroguaiaretic acid, trihydroxybutyrophenone, uric acid and the derivatives thereof, mannose and the derivatives thereof, zinc and the derivatives thereof (for example ZnO, ZnSO₄), selenium and the derivatives thereof (for example selenium methionine), superoxide dismutase, stilbenes and the derivatives thereof (for example stilbene oxide, trans-stilbene oxide) and derivatives suitable according to the invention (salts, esters, ethers, sugars, nucleotides, nucleosides, peptides and lipids) of these stated active ingredients or antioxidatively active extracts or fractions of plants such as for example green tea, rooibos, honeybush, grape, rosemary, sage, melissa, thyme, lavender, olive, oats, cocoa, ginkgo, ginseng, liquorice, honeysuckle, Sophora, Pueraria, Pinus, citrus, Phyllanthus emblica or St. John's wort, grapeseed, wheat germ, Indian gooseberry.

Coenzymes, such as for example coenzyme Q10, plastoquinone, menaquinone, ubiquinols 1-10, ubiquinones 1-10 or derivatives of these substances are furthermore suitable.

The amount of antioxidants (one or more compounds) in the formulations according to the invention preferably amounts to 0.01 to 20 wt. %, particularly preferably 0.05 to 10 wt. %, in particular 0.2 to 5 wt. %, relative to the total weight of the formulation.

If vitamin E and/or the derivatives thereof constitute the antioxidant(s), it is advantageous to select the particular concentrations thereof from the range of 0.001 to 10 wt. %, relative to the total weight of the formulation.

If vitamin A or vitamin A derivatives, or carotenes or the derivatives thereof constitute the antioxidant(s), it is advantageous to select the particular concentrations thereof from the range of 0.001 to 10 wt. %, relative to the total weight of the formulation.

Formulations according to the invention may also contain preservatives. Preservatives which may be used are: any antioxidants suitable for or conventional in cosmetic (for example dermatological) and/or therapeutic applications, conventional preservatives (for example formaldehyde, glutardialdehyde, parabens (for example methyl-, ethyl-, propyl- and butylparaben), dibromodicyanobutane, imidazolidinylureas (“Germall”), isothiazolinones (“Kathon”), methylchlorothiazolidine, methylthiazolidine, organic acids (for example benzoic acid, sorbic acid, salicylic acid) and the salts and esters thereof, propionic acid and formic acid and the salts thereof glycols for example propylene glycol, 1,2-dihydroxyalkanes), plant-derived preservation aids such as for example lantadin A, caryophyllene, hesperidin, diosmin, phellandrene, pigenin, quercetin, hypericin, aucubin, diosgenin, plumbagin, corlilagin etc.

It may furthermore be advantageous to use antiirritants in formulations according to the invention. Antiirritants may here be any active ingredients which have an antiinflammatory action or relieve erythema and itching and are suitable for or conventional in cosmetic (for example dermatological) and/or therapeutic applications. Preferred substances are any which reduce the amount of cytokines, interleukins, prostaglandins and/or leukotrienes in cells and tissues.

Active ingredients which have an antiinflammatory action or relieve erythema and itching which are advantageously used are steroidal antiinflammatory substances of the corticosteroid type, such as for example hydrocortisone, dexamethasone, dexamethasone phosphate, methylprednisolone or cortisone, it being possible to extend this list by the addition of further steroidal antiinflammatory drugs. Nonsteroidal antiinflammatory drugs may also be used. Examples which may be mentioned are: oxicams such as piroxicam or tenoxicam; salicylates such as aspirin, disalcid, solprin or fendosal; acetic acid derivatives such as diclofenac, fenclofenac, indomethacin, sulindac, tolmetin, or clindanac; fenamates such as mefenamic, meclofenamic, flufenamic or niflumic; propionic acid derivatives such as ibuprofen, naproxen, benoxaprofen or pyrazoles such as phenylbutazone, oxyphenylbutazone, feprazone or azapropazone. Alternatively, natural substances which have an antiinflammatory action or relieve erythema and itching may be used. Plant extracts may also be used, specifically highly active plant extract fractions and high purity active substances isolated from plant extracts. Particular preference is given to extracts, fractions and active substances from chamomile, aloe vera, Commiphora species, Rubia species, Echinacea species, willows, willowherb, oats, black and green tea, gingko, coffee, pepper, blackcurrants, tomato, vanilla, almonds, together with pure substances such as inter alia bisabolol, apigenin-7-glucoside, boswellia acid, phytosterols, glycyrrhizinic acid, glabridin, or licochalcone A.

The amount of antiirritants (one or more compounds) in the formulations according to the invention preferably amounts to 0.01 to 20 wt. %, particularly preferably 0.03 to 10 wt. %, in particular 0.05 to 5 wt. %, relative to the total weight of the formulation.

The formulations according to the invention (in particular topical cosmetic formulations) may furthermore contain moisture-retaining regulators and osmolytes. The following substances are examples of substances used as moisture-retaining regulators (“moisturizers”): sodium lactate, urea, alcohols (in particular 1,2-pentanediol, 1,2-hexanediol, 1,2-octanediol, 1,2-decanediol and mixtures thereof), sorbitol, glycerol, propylene glycol, collagen, elastin or hyaluronic acid, diacyl adipates, petroleum jelly, ectoin, urocaninic acid, lecithin, pantheol, phytantriol, lycopene, algae extract, ceramides, cholesterol, glycolipids, chitosan, chondroitin sulfate, polyamino acids and polyamino sugars, lanolin, lanolin esters, amino acids, alpha-hydroxy acids, (for example citric acid, lactic acid, malic acid) and the derivatives thereof, sugars (for example inositol), alpha-hydroxy-fatty acids, phytosterols, triterpene acids such as betulinic acid or ursolic acid, algae extracts. Osmolytes which may, for example, be used are is sugar alcohols (myo-inositol, mannitol, sorbitol), quaternary amines such as taurine, choline, betaine, betaine/glycine, ectoin, diglycerol phosphate, phosphorylcholine, glycerophosphoryicholine, amino acids such as glutamine, glycine, alanine, glutamate, aspartate or proline, phosphatidyl choline, phosphatidyl inositol, inorganic phosphates, and polymers of the stated compounds such as proteins, peptides, polyamino acids and polyols.

The formulations according to the invention (for example topical cosmetic formulations) furthermore advantageously contain antimicrobial active ingredients. Examples which may be mentioned are:

Aryl- or aryloxy-substituted, unbranched or mono- and poly-alkyl-branched saturated or mono- to penta-unsaturated (up to five double or triple bonds, also mixed ene/yne compounds) fatty alcohols, fatty aldehydes and fatty acids of chain lengths C₂ to C₄₀.

Aryl- or aryloxy-substituted unbranched or mono- and poly-alkyl-branched saturated or mono- to penta-unsaturated (up to five double or triple bonds, also mixed ene/yne compounds) alkanediols, dialdehydes and dicarboxylic acids of chain lengths C₂ to C₄₀, with chain lengths of C₄ to C₁₂ being particularly preferred.

Mono- and oligoglycerides (up to 4 glycerol units) of aryl- or aryloxy-substituted unbranched or mono- and poly-alkyl-branched saturated or mono- to penta-unsaturated (up to five double or triple bonds, also mixed ene/yne compounds) fatty alcohols (mono- and oligoglycerol monoalkyl ethers), fatty acids (mono- and oligoglycerol monoalkyl esters), alkanediols (mono- and oligoglycerol monoalkyl ethers; bis(mono-/oligoglyceryl) alkyl diethers) and dicarboxylic acids (mono- and oligoglycerol monoalkyl esters; bis(mono-/oligoglyceryl) alkyl diesters) of chain lengths C₂ to C₄₀.

Fatty acid esters of unbranched or mono- and poly-alkyl-branched saturated or mono- to penta-unsaturated (up to five double or triple bonds, also mixed ene/yne compounds), optionally also of aryl- or aryloxy-substituted carboxylic acids of chain lengths C₂ to C₄₀ with unbranched or mono- and poly-alkyl-branched saturated or mono- to penta-unsaturated (up to five double or triple bonds, also mixed ene/yne compounds), optionally also of aryl- or aryloxy-substituted mono- to hexahydric fatty alcohols of chain lengths C₂ to C₄₀.

Plant and animal fatty acid fractions containing unbranched or mono- and poly-alkyl-branched saturated or mono- to penta-unsaturated (up to five double or triple bonds, also mixed ene/yne compounds), fatty alcohols, fatty aldehydes and fatty acids of chain lengths C₂ to C₄₀ (for example coconut fatty acid, palm kernel fatty acids, wool wax acids).

Mono- and oligoglycerides of lanolin, of lanolin alcohols and lanolin acids (for example glyceryl lanolate, Neocerit), glycyrrhetinic acid and derivatives (for example glycyrrhetinyl stearate), natural and synthetic cardenolides (for example digitoxin, digoxin, digoxigenin, gitoxigenin, strophanthin and strophanthidine), natural and synthetic bufadienolides (for example scillaren A, scillarenin and bufotalin), sapogenins and steroid sapogenins (for example amyrins, oleanolic acid, digitonin, gitogenin, tigogenin and diosgenin), steroid alkaloids of plant and animal origin (for example tomatidine, solanine, solanidine, conessine, batrachotoxin and homobatrachotoxin).

Mono- and poly-halogenated nitriles, dinitriles, trinitriles or tetranitriles.

Mono- and oligohydroxy fatty acids of chain lengths C₂ to C₂₄ (for example lactic acid, 2-hydroxypalmitic acid), the oligomers and/or polymers thereof and plant and animal raw materials containing the same.

Acyclic terpenes: terpene hydrocarbons (for example ocimene, myrcene), terpene alcohols (for example geraniol, linalool, citronellol), terpene aldehydes and ketones (for example citral, pseudoionone, beta-ionone); monocyclic terpenes: terpene hydrocarbons (for example terpinene, terpinolene, limonene), terpene alcohols (for example terpineol, thymol, menthol), terpene ketones (for example pulegone, carvone); bicyclic terpenes: terpene hydrocarbons (for example carane, pinane, bornane), terpene alcohols (for example borneol, isoborneol), terpene ketones (for example camphor); sesquiterpenes: acyclic sesquiterpenes (for example farnesol, nerolidol), monocyclic sesquiterpenes (for example bisabolol), bicyclic sesquiterpenes (for example cadinene, selinene, vetivazulene, guaiazulene), tricyclic sesquiterpenes (for example santalene), diterpenes (for example phytol), tricyclic diterpenes (for example abietic acid), triterpenes (squalenoids; for example squalene), tetraterpenes.

Ethoxylated, propoxylated or mixed ethoxylated/propoxylated cosmetic fatty alcohols, fatty acids and fatty acid esters of chain lengths C₂ to C₄₀ with 1 to 150 EO and/or PO units.

Antimicrobial peptides and proteins with an amino acid count of 4 to 200, for example skin antimicrobial peptides (SAPs), lingual antimicrobial peptides so (LAPs), human beta-defensins (in particular h-BD1 and h-BD2), lactoferrins and the hydrolysates thereof and peptides obtained therefrom, bactericidal/permeability-increasing proteins [BPIs], cationic microbial proteins [CAPs], lysozyme.

Highly suitable carbohydrates or “carbohydrate derivatives”, which for succinctness' sake are intended also to fall within the term “carbohydrates”, are sugars and substituted sugars or compounds containing sugar residues. Sugars in particular also in each case include the deoxy and dideoxy forms, N-acetylgalactosamine-, N-acetylglucosamine- and sialinic acid-substituted derivatives as well as sugar esters and ethers. Preferred substances are
a) monosaccharides, among which in particular pentoses and hexoses,
b) disaccharides, among which in particular sucrose, maltose, lactobiose,
c) oligosaccharides, among which in particular tri- and tetrasaccharides and
d) polysaccharides, among which in particular starch, glycogen, cellulose, dextran, tunicin, inulin, chitin, in particular chitosan, chitin hydrolysates, alginic acid and alginates, plant gums, mucous secretions, pectins, mannans, galactans, xylans, araban, polyoses, chondroitin sulfates, heparin, hyaluronic acid and glycosaminoglycans, hemicelluloses, substituted cellulose and substituted starch, in particular in each case the hydroxyalkyl-substituted polysaccharides.

Amylose, amylopectin, xanthan, alpha-, beta- and gamma-dextrin are particularly suitable. The polysaccharides may consist, for example, of 4 to 1,000,000, in particular 10 to 100,000 monosaccharides. Preferably in each case those chain lengths are selected which ensure that the active ingredient is soluble in the particular formulation or may be incorporated therein.

Sphingolipids such as sphingosine; N-monoalkylated sphingosines; N,N-dialkylated sphingosines; sphingosine 1-phosphate; sphingosine 1-sulfate; psychosine (sphingosine beta-D-galactopyranoside); sphingosylphosphorylcholine; lysosulfatides (sphingosylgalactosyl sulfate; lysocerebroside sulfate); lecithin; sphingomyelin; sphinganine.

“Natural” antibacterial active ingredients may also be used, these mainly being essential oils. Typical oils with an antibacterial action are for example oils obtained from aniseed, lemon, orange, rosemary, wintergreen, clove, thyme, lavender, hops, citronella, wheat, lemon grass, cedar wood, cinnamon, geranium, sandalwood, violet, eucalyptus, peppermint, gum benzoin, basil, fennel, menthol and Ocotea, Origanum, Hydrastis carradensis, Berberidaceae, Ratanhiae or Curcuma longa.

Important antimicrobial active substances, which may be found in essential oils are, for example, anethole, catechol, camphene, carvacrol, eugenol, eucalyptol, ferulic acid, farnesol, hinokitiol, tropolone, limonene, menthol, methyl salicylate, thymol, terpineol, verbenone, berberine, curcumin, caryophyliene oxide, nerolidol, geraniol.

Mixtures of the stated active systems or active ingredients and active ingredient combinations which contain these active ingredients may also be used.

The amount of antimicrobial active ingredients in the formulations preferably amounts to 0.01 to 20 wt. %, relative to the total weight of the formulations, particularly preferably 0.05 to 10 wt. %.

The formulations according to the invention (in particular cosmetic, including dermatological formulations) may contain deodorants, i.e. active ingredients with a deodorizing and antiperspirant action. These include for example odor masking agents, such as conventional perfume constituents, antiperspirants based on aluminum, zirconium or zinc salts, odor absorbers, for example the phyllosilicates described in published patent application DE-P 40 09 347, of these in particular montmorillonite, kaolinite, nontronite, saponite, hectorite, bentonite, smectite, furthermore for example zinc salts of ricinoleic acid. These also include bactericidal or bacteriostatic deodorizing substances, such as for example hexachlorophene, 2,4,4′-trichloro-2′-hydroxydiphenyl ether (Irgasan), 1,6-di-(4-chlorophenyldiguanidino)hexane (chlorhexidine), 3,4,4′-trichlorocarbanilide, and the active agents described in published patent applications DE-37 40 186, DE-39 38 140, DE-42 04 321, DE-42 29 707, DE-42 29 737, DE-42 37 081, DE-43 09 372, DE-43 24 219, and contain cationically active substances, such as for example quaternary ammonium salts and odor absorbers, such as for example Grillocin® (combination of zinc ricinoleate and various additives) or triethyl citrate, optionally in combination with ion-exchange resins.

The amount of deodorizing and/or antiperspirant active ingredients in the formulations preferably amounts to 0.01 to 20 wt. %, relative to the total weight of the formulations, particularly preferably 0.05 to 10 wt. %.

The formulations (in particular cosmetic formulations) according to the invention may, in particular if crystalline or microcrystalline solids, for example inorganic micropigments, are to be incorporated onto the formulations, also contain anionic, cationic, nonionic and/or amphoteric surfactants.

Anionic surfactants generally comprise carboxylate, sulfate or sulfonate groups as the functional groups. In an aqueous solution they form negatively charged organic ions in an acidic or neutral medium. Cationic surfactants are almost exclusively characterized by the presence of a quaternary ammonium group. In an aqueous solution they form positively charged organic ions in an acidic or neutral medium. Amphoteric surfactants contain both anionic and cationic groups and in an aqueous solution accordingly behave like anionic or cationic surfactants depending on the pH value. In a strongly acidic medium they have positive charge and in an alkaline medium a negative charge. In the neutral pH range, on the other hand, they are zwitterionic. Typical examples of nonionic surfactants are polyether chains. Nonionic surfactants do not form ions in an aqueous medium.

### A. Anionic Surfactants

Anionic surfactants which may advantageously be used are acyl amino acids (and the salts thereof, such as
acyl glutamates, for example sodium acyl glutamate, di-TEA-palmitoyl aspartate and sodium capryl/capric glutamate,
acyl peptides, for example palmitoyl-hydrolyzed milk protein, sodium cocoyl-hydrolyzed soy protein and sodiumipotassium cocoyl-hydrolyzed coliagen,
sarcosinates, for example myristoyl sarcosine, TEA lauroyl sarcosinate, sodium lauroyl sarcosinate and sodium cocoyl sarcosinate,
taurates, for example sodium lauroyl taurate and sodium methylcocoyl taurate,
acyl lactylates, lauroyl lactylate, caproyl lactylate
alaninates
   carboxylic acids and derivatives, such as
   for example lauric acid, aluminum stearate, magnesium alkanolate and zinc undecylenate,
ester carboxylic acids, for example calcium stearoyl lactylate, laureth-6 citrate and sodium PEG 4 lauramide carboxylate,
ether carboxylic acids, for example sodium laureth-13 carboxylate and sodium PEG-6 cocamide carboxylate,
   phosphoric acid esters and salts, such as for example DEA oleth-10 phosphate and dilaureth-4 phosphate,
   sulfonic acids and salts, such as
acyl isethionates, for example sodium/ammonium cocoyl isethionate,
alkylaryl sulfonates,
alkyl sulfonates, for example sodium cocomonoglyceride sulfate, sodium C₁₂₋₁₄ olefin sulfonate, sodium lauryl sulfoacetate and magnesium PEG-3 cocamide sulfate,
sulfosuccinates, for example dioctyl sodium sulfosuccinate, disodium laureth sulfosuccinate, disodium lauryl sulfosuccinate and disodium undecyleneamido MEA sulfosuccinate
   and
   sulfuric acid esters, such as
alkyl ether sulfate, for example sodium, ammonium, magnesium, MIPA, TIPA laureth sulfate, sodium myreth sulfate and sodium C12-13 pareth sulfate,
alkyl sulfates, for example sodium, ammonium and TEA lauryl sulfate.

### B. Cationic Surfactants

Cationic surfactants which may advantageously be used are
alkylamines,
alkylimidazoles,
ethoxylated amines and
quaternary surfactants.

RNH₂CH₂CH₂COO⁻ (at pH=7)

RNHCH₂CH₂COO— B⁺ (at pH=12) B⁺=any desired cation, for example Na⁺
ester quats

Quaternary surfactants contain at least one N atom which is covalently bonded with 4 alkyl or aryl groups. This results, irrespective of pH value, in a positive charge. Alkyl betaine, alkylamidopropyl betaine and alkylamidopropyl hydroxysultaine are advantageous. The cationic surfactants used may furthermore preferably be selected from the group of quaternary ammonium compounds, in particular benzyltrialkylammonium chlorides or bromides, such as for example benzyldimethylstearylammonium chloride, furthermore alkyltrialkylammonium salts, for example cetyltrimethylammonium chloride or bromide, alkyldimethylhydroxyethylammonium chlorides or bromides, dialkyldimethyl-ammonium chlorides or bromides, alkylamidoethyltrimethylammonium ether sulfates, alkylpyridinium salts, for example lauryl- or cetylpyrimidinium chloride, imidazoline derivatives and compounds with a cationic nature such as amine oxides, for example alkyl dimethyl amine oxides or alkyl aminoethyl dimethyl amine oxides. Cetyltrimethylammonium salts may in particular advantageously be used.

### C. Amphoteric Surfactants

Amphoteric surfactants which may advantageously be used are
acyl/dialkyl ethylenediamine, for example sodium acyl amphoacetate, disodium acyl amphodipropionate, disodium alkyl amphodiacetate, sodium acyl amphohydroxypropylsulfonate, disodium acyl amphodiacetate and sodium acyl amphopropionate,
N-Alkyl amino acids, for example aminopropyl alkylglutamide, alkylaminopropionic acid, sodium alkylimidodipropionate and lauroamphocarboxyglycinate.

### D. Nonionic Surfactants

Nonionic surfactants which may advantageously be used are
alcohols,
alkanolamides, such as cocamide MEA/DEA/MIPA,
amine oxides, such as cocoamidopropyl amine oxide,
esters which arise by esterification of carboxylic acids with ethylene oxide, glycerol, sorbitan or other alcohols,
ethers, for example ethoxylated/propoxylated alcohols, ethoxylated/propoxylated esters, ethoxylated/propoxylated glycerol esters, ethoxylated/propoxylated cholesterols, ethoxylated/propoxylated triglyceride esters, ethoxylated/propoxylated lanolin, ethoxylated/propoxylated polysiloxanes, propoxylated POE ethers and alkyl polyglycosides such as lauryl glucoside, decyl glycoside and coco glycoside.
sucrose (saccharose) esters, ethers
polyglycerol esters, diglycerol esters, monoglycerol esters
methylglucose esters, esters of hydroxy acids

It is furthermore advantageous to use a combination of anionic and/or amphoteric surfactants with one or more nonionic surfactants.

The surface-active substance (surfactant) or the combination of surface-active substances may be present in the formulations according to the invention in a concentration of between 1 and 98 wt. %, relative to the total weight of the formulations.

Cosmetic (for example dermatological) or therapeutic formulations according to the invention which contain compounds of the formula (I) according to the invention or to be used according to the invention, may also be present as emulsions.

The oil phase (lipid phase) in the formulations according to the invention (in particular topical cosmetic formulations) may advantageously be selected from the following group of substances:
mineral oils (advantageously paraffin oil), mineral waxes
fatty oils, fats, waxes and other natural and synthetic fatty bodies, preferably esters of fatty acids with alcohols with a small number of C atoms, for example with isopropanol, propylene glycol or glycerol, or esters of fatty alcohols with alkanoic acids with a small number of C atoms or with fatty acids;
alkyl benzoates (for example mixtures n-dodecyl, n-tridecyl, n-tetradecyl or n-pentadecyl benzoate);
cyclic or linear silicone oils such as dimethylpolysiloxanes, diethylpolysiloxanes, diphenylpolysiloxanes and mixed forms thereof.

Esters (natural or synthetic) may advantageously be used, in particular (a) esters prepared from saturated and/or unsaturated branched and/or unbranched alkanecarboxylic acids of a chain length of 3 to 30 C atoms and saturated and/or unsaturated, branched and/or unbranched alcohols of a chain length of 3 to 30 C atoms, (b) esters prepared from aromatic carboxylic acids and saturated and/or unsaturated, branched and/or unbranched alcohols of a chain length of 3 to 30 C atoms. Preferred ester oils are isopropyl myristate, isopropyl palmitate, isopropyl stearate, isopropyl oleate, n-butyl stearate, n-hexyl laurate, n-decyl laurate, n-decyl oleate, isooctyl stearate, isononyl stearate, isononyl isononanoate, 3,5,5-dimethylhexyl 3,5,5-trimethylhexanoate, 2-ethylhexyl isononanoate, 2-ethylhexyl 3,5,5-trimethylhexanoate, 2-ethylhexyl 2-ethylhexanoate, cetearyl 2-ethylhexanoate, 2-ethylhexyl palmitate, 2-ethylhexyl laurate, 2-hexyldecyl stearate, 2-octyidecyl palmitate, 2-octyidodecyl palmitate, oleyl oleate, oleyl erucate, erucyl oleate, erucyl erucate and synthetic, semisynthetic and natural mixtures of such esters, for example jojoba oil.

The oil phase may furthermore advantageously be selected from the group of branched and unbranched hydrocarbons and hydrocarbon waxes, silicone oils, dialkyl ethers, the group of saturated or unsaturated, branched or unbranched alcohols, and fatty acid triglycerides, specifically the triglycerol esters of saturated and/or unsaturated, branched and/or unbranched alkanecarboxylic acids of a chain length of 8 to 24, in particular 12 to 18 C atoms. The fatty acid triglycerides may advantageously be selected from the group of synthetic, semisynthetic and natural oils, for example triglycerides of capric or caprylic acid, apricot kernel oil, avocado oil, cottonseed oil, borage seed oil, thistle oil, peanut oil, gamma-oryzanol, rose hip seed oil, hemp oil, hazelnut oil, blackcurrant seed oil, coconut oil, cherry stone oil, salmon oil, linseed oil, maize germ oil, macadamia oil, almond oil, evening primrose oil, mink oil, olive oil, palm oil, palm kernel oil, pecan oil, peach stone oil, pistachio oil, rapeseed oil, rice germ oil, castor oil, safflower oil, sesame oil, soy oil, sunflower oil, tea tree oil, grapeseed oil or wheat germ oil, and other similar substances. Any desired blends of such oil and wax components may also advantageously be used. In many cases, it is also advantageous to use waxes, for example cetyl palmitate, as the sole lipid component of the oil phase, the oil phase is advantageously selected from the group which consists of 2-ethylhexyl isostearate, octyidodecanol, isotridecyl isononanoate, isoeicosane, 2-ethylhexyl cocoate, C₁₂₋₁₅ alkyl benzoate, caprylic/capric acid triglyceride and dicaprylyl ether. Particularly advantageous mixtures are those prepared from C₁₂₋₁₅ alkyl benzoate, and 2-ethylhexyl isostearate, mixtures of C₁₂₋₁₅ alkyl benzoate and isotridecyl isononanoate and mixtures of C₁₂₋₁₅ alkyl benzoate, 2-ethylhexyl isostearate and isotridecyl isononanoate. The hydrocarbons paraffin oil, squalane and squalene may also advantageously be used. The oil phase may furthermore advantageously comprise a content of cyclic or linear silicone oils or completely consist of such oils, wherein it is however preferred to use an additional content of other oil phase components apart from the silicone oil or silicone oils. Cyclomethicone (for example decamethylcyclopentasiloxane) may advantageously be used as silicone oil. Other silicone oils may, however, advantageously be used, for example undecamethylcyclotrisiloxane, polydimethylsiloxane and poly(methylphenylsiloxane). Mixtures of cyclomethicone and isotridecyl isononanoate, and of cyclomethicone and 2-ethylhexyl isostearate are furthermore particularly advantageous.

The aqueous phase of formulations (in particular topical cosmetic formulations) according to the invention which assume emulsion form may advantageously comprise: alcohols, diols or polyols small number of C atoms, and the ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and similar products, furthermore alcohols with a small number of C atoms, for example ethanol, isopropanol, 1,2-propanediol, glycerol and in particular one or more thickeners, which may advantageously be selected from the group of silicon dioxide, aluminum silicates such as for example bentonite, polysaccharides or the derivatives thereof, for example hyaluronic acid, guar flour, xanthan gum, hydroxypropylmethylcellulose, or allulose derivatives, particularly advantageously from the group of polyacrylates, preferably a polyacrylate from the group of “carbopols”, for example carbopols of types 980, 981, 1382, 2984, 5984, in each case individually or in combination, or from the group of polyurethanes, furthermore alpha- or beta-hydroxy acids, preferably lactic acid, citric acid or salicylic acid, as well as emulsifiers, which may advantageously be selected from the group of ionic, nonionic, polymeric, phosphate-containing and zwitterionic emulsifiers,

Formulations according to the invention which assume emulsion form advantageously comprise one or more emulsifiers. O/W emulsifiers may for example advantageously be selected from the group of polyethoxylated or polypropoxylated or polyethoxylated and polypropoxylated products, for example:
fatty alcohol ethoxylates
ethoxylated wool wax alcohols,
polyethylene glycol ethers of the general formula R—O—(—CH₂—CH₂—O—)ₙ—R′,
fatty acid ethoxylates of the general formula R—COO—(—CH₂—CH₂—O—)ₙ—H,
etherified fatty acid ethoxylates of the general formula

R—COO—(—CH₂—CH₂—O—)ₙ—R′,
esterified fatty acid ethoxylates of the general formula

R—COO—(—CH₂—CH₂—O—)ₙ—C(O)—R′,
polyethylene glycol glycerol fatty acid esters
ethoxylated sorbitan esters
cholesterol ethoxylates
ethoxylated triglycerides
alkyl ether carboxylic acids of the general formula

R—COO—(—CH₂—CH₂—O—)ₙ—OOH, n representing a number from 5 to 30,
polyoxyethylene sorbitol fatty acid esters,
alkyl ether sulfates of the general formula R—O—(—CH₂—CH₂—O—)ₙ—SO₃—H
fatty alcohol propoxylates of the general formula R—O—(—CH₂—CH(CH₃)—O—)ₙ—H
polypropylene glycol ethers of the general formula

R—O—(—CH₂—CH(CH₃)—O—)ₙ—R′
propoxylated wool wax alcohols,
etherified fatty acid propoxylates R—COO—(—CH₂—CH(CH₃)—O—)ₙ—R′
esterified fatty acid propoxylates of the general formula

R—COO—(—CH₂—CH(CH₃)—O—)ₙ—C(O)—R′
fatty acid propoxylates of the general formula

R—COO—(—CH₂—CH(CH₃)—O—)ₙ—H,
polypropylene glycol glycerol fatty acid esters
propoxylated sorbitan esters
cholesterol propoxylates
propoxylated triglycerides
alkyl ether carboxylic acids of the general formula

R—O—(—CH₂—CH(CH₃)—O—)ₙ—CH₂—COOH,
alkyl ether sulfates or the acids, on which these sulfates are based, of the general formula R—O—(—CH₂—CH(CH₃)—O—)ₙ—SO₃—H,
fatty alcohol ethoxylates/propoxylates of the general formula R—O—Xₙ—Yₘ—H
polypropylene glycol ethers of the general formula R—O—Xₙ—Yₘ—R′
etherified fatty acid propoxylates of the general formula R—COO—Xₙ—Yₘ—R′
fatty acid ethoxylates/propoxylates of the general formula R—COO—Xₙ—Yₘ—H.

According to the invention, the polyethoxylated or polypropoxylated or polyethoxylated and polypropoxylated O/W emulsifiers are particularly advantageously selected from the group of substances with HLB values of 11 to 18, very particularly advantageously with HLB values of 14.5 to 15.5, provided that the O/W emulsifiers comprise saturated residues R and R′. If the O/W emulsifiers comprise unsaturated residues R and/or R′, or if isoalkyl derivatives are present, the preferred HLB value of such emulsifiers may also be lower or higher.

It is advantageous to select the fatty alcohol ethoxylates from the group of ethoxylated stearyl alcohols, cetyl alcohols, cetylstearyl alcohols (cetearyl alcohols). In particular, the following are preferred:

polyethylene glycol (n) stearyl ether (steareth-n) with n=13-20,

polyethylene glycol (n) cetyl ether (ceteth-n) with n=13-20,

polyethylene glycol (n) isocetyl ether (isoceteth-n) with n=13-20,

polyethylene glycol (n) cetylstearyl ether (ceteareth-n) with n=13-20,

polyethylene glycol (m) isostearyl ether (isosteareth-m) with m=12-20,

polyethylene glycol (k) oleyl ether (oleth-k) with k=12-15,

polyethylene glycol (12) lauryl ether (laureth-12),

polyethylene glycol (12) isolauryl ether (isolaureth-12).

It is furthermore advantageous to select the fatty acid ethoxylates from the following group:

polyethylene glycol (n) stearate, with n=20-25

polyethylene glycol (m) isostearate with m=12-25

polyethylene glycol (k) oleate with k=12-20

Sodium laureth 11 carboxylate may advantageously be used as an ethoxylated alkyl ether carboxylic acid or the salt thereof. Sodium laureth 1-4 sulfate may advantageously be used as an alkyl ether sulfate. Polyethylene glycol (30) cholesteryl ether may advantageously be used as an ethoxylated cholesterol derivative. Polyethylene glycol (25) soy sterol has also proved effective.

Polyethylene glycol (60) evening primrose glycerides may advantageously be used as ethoxylated triglycerides.

It is furthermore of advantage to select the polyethylene glycol glycerol fatty acid esters from the group of polyethylene glycol (n) glyceryl laurate with n=20-23, polyethylene glycol (6) glyceryl caprate/caprinate, polyethylene glycol (20) glyceryl oleate, polyethylene glycol (20) glyceryl isostearate, polyethylene glycol (18) glyceryl oleate/cocoate.

It is likewise favorable to select the sorbitan esters from the group of polyethylene glycol (20) sorbitan monolaurate, polyethylene glycol (20) sorbitan monostearate, polyethylene glycol (20) sorbitan monoisostearate, polyethylene glycol (20) sorbitan monopalmitate, polyethylene glycol (20) sorbitan monooleate.

Advantageous W/O emulsifiers which may be used are fatty alcohols with 8 to 30 carbon atoms, monoglycerol esters of saturated and/or unsaturated, branched and/or unbranched alkanecarboxylic acids of a chain length of 8 to 24, in particular 12 to 18 C atoms, diglycerol esters of saturated and/or unsaturated, branched and/or unbranched alkanecarboxylic acids of a chain length of 8 to 24, in particular 12 to 18 C atoms, monoglycerol ethers of saturated and/or unsaturated, branched and/or unbranched alcohols of a chain length of 8 to 24, in particular 12 to 18 C atoms, diglycerol ethers of saturated and/or unsaturated, branched and/or unbranched alcohols of a chain length of 8 to 24, in particular 12 to 18 C atoms, propylene glycol esters of saturated and/or unsaturated, branched and/or unbranched alkanecarboxylic acids of a chain length of 8 to 24, in particular 12 to 18 C atoms, and sorbitan esters of saturated and/or unsaturated, branched and/or unbranched alkanecarboxylic acids of a chain length of 8 to 24, in particular 12 to 18 C atoms.

Particularly advantageous W/O emulsifiers are glyceryl monostearate, glyceryl monoisostearate, glyceryl monomyristate, glyceryl monooleate, diglyceryl monostearate, diglyceryl monoisostearate, propylene glycol monostearate, propylene glycol monoisostearate, propylene glycol monocaprylate, propylene glycol monolaurate, sorbitan monoisostearate, sorbitan monolaurate, sorbitan monocaprylate, sorbitan monoisooleate, sucrose distearate, cetyl alcohol, stearyl alcohol, arachidyl alcohol, behenyl alcohol, isobehenyl alcohol, selachyl alcohol, chimyl alcohol, polyethylene glycol (2) stearyl ether (steareth-2), glyceryl monolaurate, glyceryl monocaprate, glyceryl monocaprylate.

Mixtures of the stated active systems may also be used.

The total amount of compounds of the formula (II), UV absorber and (further) skin-lightening agents in the formulations according to the invention preferably amounts to 0.0001 to 20 wt. %, relative to the total weight the formulation, particularly preferably to 0.0005 to 15 wt. %.

The topical formulations according to the invention are used by being applied in a sufficient amount onto the skin and/or hair in the manner conventional for cosmetics.

The invention is illustrated in greater detail below with reference to Examples, without there being any intention of the Examples' limiting the scope of protection defined by the claims. Unless otherwise stated, all stated values relate to weight.

### EXAMPLE 1

### Production of the Compound of the Formula (X)

1 g (6.15 mmol) of isoeugenol was introduced into a mixture of 1500 ml of water and 100 ml of acetone and combined at room temperature (approx. 20° C.) with 11 mg of horseradish peroxidase (Sigma Aldrich). A mixture of 10 ml of water and 0.21 ml of 50% strength hydrogen peroxide (3.1 mmol) was then added dropwise. After a post-reaction time of approx. 3 h at room temperature (approx. 20° C.), the reaction was terminated and the organic phase separated by means of repeated extraction with an organic solvent. After evaporation, 850 mg of residue were obtained, around 80% of which was the compound of the formula (X). The residue was chromatographed on silica gel (hexane/ethyl acetate: 2/1 (vol/vol)) for analytical purposes, 350 mg of the high purity compound of the formula (X) being obtained (purity >98%). The structure of the compound of formula (X) was confirmed by means of MS and NMR.

### EXAMPLE 2

### Detection of UVB-Triggered AhR Induction in Human Keratinocytes and the Action of AhR Antagonists

### Cell Culture and Irradiation

HaCaT keratinocytes were cultured in DMEM medium with 10% fetal calf serum. The cells were irradiated with UVB radiation in PBS (phosphate-buffered saline). For UVB irradiation, we used a TL20W/12RS lamp which contains four parallel tubes (Philips, Eindhoven, Netherlands) and emits the majority of its energy in the UVB range (290-320 nm). The emission peak of the lamp is at 310 nm. Control cells were subjected to identical treatment, but were not irradiated. In order to inhibit the AhR, the cells were treated with the test substance 1 h before irradiation.

Transfection of HaCaT Cells with the pEGFP-AhR Plasmid

HaCaT cells were plated out onto chambered microscope slides with a cell density of 5×10⁴ cells/chamber. Some were pretreated with the test substance for 1 h. After 24 h, said cells were transfected with the pEGFP-AhR plasmid by means of the FuGene 6 transfection reagent (Roche, Mannheim, Germany) in accordance with the manufacturer's instructions After a further 24 h, transfected cells were irradiated with 100 μm² of UVB. After 40 min, the cells were fixed for 10 min with 4% paraformaldehyde and washed with PBS. The microscope slides were dried and mounted with Vectashield Mounting Medium (Vector Laboratories, Burlingame, Calif., USA). The AhR coupled to the GFP was visualized by means of a fluorescence microscope (Olympus, Hamburg, Germany) and photographed with a ColorViewXS digital camera (Olympus).

RNA Preparation, cDNA Synthesis and Real Time RT-PCR

HaCaT cells were irradiated with 100 J/m² of UVB. Some cells were pretreated with the test substance 1 h before irradiation. After 4 h, the RNA was prepared using the RNeasy kit (Qiagen, Hilden, Germany) in accordance with the manufacturer's instructions. Reverse transcription was carried out as described (Arch. Toxicol (2005) PMID 16205913). PCR fragments were amplified by means of real-time PCR in a LightCycler (Roche, Mannheim, Germany). The PCR mix was made up of 1/10 volume of QuantiTec® SYBR Green PCR Master Mixes F (Qiagen, Hilden, Germany), 0.5 μM of the particular primer, 2 μl of cDNA and DEPC-treated (diethyl pyrocarbonate-treated) H₂O in a final volume of 20 μl. PCR began with an initial 15 min of heating to 95° C. to activate the DNA polymerase. PCR conditions were as follows: 40 cycles of 15 sec 94° C. for denaturation, 25 sec 60° C. for primer attachment, 30 sec 72° C. for extension and 2 sec 72° C. for fluorescence measurement. PCR primers for human CYP1A1 had the following sequences: 5′-TAGACACTGATCTGGCTGCAG for the forwards primer and 5′-GGGAAGGCTCCATCAGCATC for the backwards primer (Cancer Res. 1990, 50, 4315), which formed a 146 bp fragment after amplification. The PCR products were quantified by means of a fragment-specific standard curve and analysis with LightCycler Software 3. Standard curves were produced with 10² to 10⁶ of CYP1A1 cDNA copies/μl and amplified as described above.

### Results

The standard, 3-methoxy-4-nitroflavone, inhibited AhR translocation into the cell nucleus in the presence and absence of UVB light and so exhibited the action expected from the literature.

Quantification of CYP1A1 by Means of RNA Preparation, cDNA Synthesis and Real Time RT-PCR

HaCaT cells were irradiated with 100 J/m² of UVB. Some cells were pretreated with the test substance 1 h before irradiation. After 4 h, the RNA was prepared using the RNeasy kit (Qiagen, Hilden, Germany) in accordance with the manufacturer's instructions. Reverse transcription was carried out as described (Arch. Toxicol (2005) PMID 16205913). PCR fragments were amplified by means of real-time PCR in a LightCycler (Roche, Mannheim, Germany). The PCR mix was made up of 1/10 volume of QuantiTect® SYBR Green PCR Master Mixes (Qiagen, Hilden, Germany), 0.5 μM of the particular primer, 2 μl of cDNA and DEPC-treated (diethyl pyrocarbonate-treated) H₂O in a final volume of 20 μl. PCR began with an initial 15 min of heating to 95° C. to activate the DNA polymerase. PCR conditions were as follows: 40 cycles of 15 sec 94° C. for denaturation, 25 sec 60° C. for primer attachment, 30 sec 72° C. for extension and 2 sec 72° C. for fluorescence measurement. PCR primers for human CYP1A1 had the following sequences: 5′-TAGACACTGATCTGGCTGCAG for the forwards primer and 5′-GGGAAGGCTCCATCAGCATC for the backwards primer (Cancer Res. 1990, 50, 4315), which formed a 146 bp fragment after amplification. The PCR products were quantified by means of a fragment-specific standard curve and analysis with LightCycler Software 3. Standard curves were produced with 102 to 106 of CYP1A1 cDNA copies/μl and amplified as described above.

TABLE 1

| | | CYP1A1 inhibition in the |
|---|---|---|
| Substance | Concentration | presence of UVB light* |
| Compound (X) | 0.0001 wt. % | 20% |

*relative to control (PBS without test substances with 0.1% DMSO, UVB- irradiated)

The compound according to the invention of the formula (X) inhibited induction of CYP1A1 as a result of AhR activation in the presence of UVB light.

### EXAMPLE 3

### Detection of Induction of CYP1A1 by AhR Activation in Murine Melanocytes

Melanocytes from mice of strain C57BL/6 were expanded in culture (medium: “melanocyte growth medium” (Promocell), 37° C., 5% CO₂). Fibroblast growth was inhibited by addition of G418 (day 10-13, 45 μg/ml). The semiconfluent cells were treated for up to 7 days with the AhR agonist FICZ in order to modulate the AhR. Untreated cells were cultured in parallel. The medium was then removed, the cells lysed, the RNA prepared as described in Example 2 and the content of CYP450 1A1 RNA determined photometrically with quantitative RT-PCR.

Result: The amount of CYP450 μl mRNA in the cells is significantly increased by this procedure in comparison with untreated cells (p<0.05, Student's t-test).
Transfection of Melanocytes with the pEGFP-AhR Plasmid

Primary mouse melanocyte cells were plated out onto chambered microscope slides. Individual chambers were treated for 1 h with the test substances. After 24 h, the cells were transfected with the pEGFP-AhR plasmid. After a further 40 min, the cells were fixed. The microscope slides were evaluated by fluorescence microscopy. Translocation of the AhR, which is coupled to the GFP and thus fluoresces green, from the cytoplasm into the cell nucleus is visible.

### Results

3-methoxy-4-nitroflavone (MNF) inhibited AhR translocation into the cell nucleus in the presence and absence of benzo[a]pyrene.

FICZ stimulated AhR translocation into the cell nucleus in the presence and absence of benzo[a]pyrene.

### EXAMPLE 4

### Modulation of Melanin Synthesis with AhR Modulators

Enhancement of Melanin Synthesis with FICZ

FICZ was generated by 60 minutes' irradiation of a 50 mM tryptophan solution with a UVB source. Semiconfluent cultures of primary mouse melanocytes (culture conditions as above) were cultured for 2 days with this solution containing FICZ. The cells were removed, lysed with 0.1% Triton-X100, centrifuged and the melanin content from the lysate pellet was determined photometrically in an ELISA reader at 405 nm after cell disruption with 1M NH₄OH (4 h, 85° C.). The blank reading, which contained only NH₄OH, was subtracted from the absorption value. In parallel, the protein content of the lysate supernatants was determined by the Bradford method. The FICZ-treated melanocytes contained significantly more melanin than the untreated cells (p<0.05, Student's t-test) (FIG. 3).

Inhibition of Melanin Synthesis with MNF

Semiconfluent primary mouse melanocytes (culture conditions as above) were further cultured for 7 days with 10 μM MNF. The cells were removed, lysed with 0.1% Triton-X100, centrifuged and the melanin content from the lysate pellet was determined photometrically in an ELISA reader at 405 nm after cell disruption with 1M NH₄OH (4 h, 85° C.). The blank reading, which contained only NH₄OH, was subtracted from the absorption value.

Result: The MNF-treated melanocytes contained significantly less melanin than the untreated cells (p<0.05, Student's t-test) (FIG. 4)

### EXAMPLE 5

### Examples of Formulations

Formulation 1: “Water in oil” emulsion with UVA/B broadband protection

Formulation 2: “Oil in water” emulsion with UVA/B broadband protection

Formulation 3: “Oil in water” emulsion with UVA/B broadband protection

Formulation 4: Oil-free sunspray with UVA/B broadband protection

Formulation 5: Balm with UVA/UVB protection

Formulation 6: Aerosol foam with UVB/UVA protection

Formulation 7: Nonaerosol foam

Formulation 8: Shampoo with UVB cell protection

Formulation 9: Hair conditioner with UVB/UVA protection

Formulation 10: O/W day cream with UVB cell protection

Formulation 11: W/O night cream with UVB cell protection

TABLE 2
Compositions of formulations according to the invention (formulations 1-11)

| RAW MATERIAL | | | | | | | | | | | | |
|---|---|---|---|---|---|---|---|---|---|---|---|---|
| NAME | | Weight % | | | | | | | | | | |
| (MANUFACTURER) | INCI | 1 | 2 | 3 | 4 | 5 | 6 | 7 | 8 | 9 | 10 | 11 |
| AhR antagonists | | | | | | | | | | | | |
| Compound (X) | | 0.2 | 5.0 | 0.1 | 0.2 | 1.0 | 0.5 | 0.1 | 1.0 | 0.2 | 1.5 | 0.4 |
| Further ingredients | | | | | | | | | | | | |
| Abil 100 ® | Dimethicone | | 1.0 | 0.3 | | | | | | | | |
| (Goldschmidt) | | | | | | | | | | | | |
| Abil 350 | Dimethicone | | | | | | | | | | 0.5 | |
| (Degussa- | | | | | | | | | | | | |
| Goldschmidt) | | | | | | | | | | | | |
| -(Alpha-)- | Bisabolol | | | | | | | | | | 0.3 | 0.2 |
| bisabolol, natural | | | | | | | | | | | | |
| (Symrise) | | | | | | | | | | | | |
| Aloe Vera Gel | Water (aqua), Aloe | | | | | | | | | | | |
| Concentrate 10/1 | barbadensis leaf juice | | | | | | | | | | | |
| (Symrise) | | | | | | | | | | | | |
| Alugel 34 TH | Aluminum stearate | | | | | | | | | | | 1.0 |
| (Baerlocher) | | | | | | | | | | | | |
| Arbutin (Sabinsa) | β-Arbutin | | | | | | | | | | 1.0 | |
| Arlypon F | Laureth-2 | | | | | | | | 2.0 | | | |
| Baysilone Oil M10 | Dimethicone | | | | | | | 1.0 | | | | |
| (GE Bayer) | | | | | | | | | | | | |
| Baysilone Oil PK | Phenyl trimethicone | | | | | 5.0 | | | | | | |
| 20 (GE Bayer) | | | | | | | | | | | | |
| Bentone Gel M | Mineral oil and | | 3.0 | | | | | | | | | |
| IO ® (Rheox) | quaternium-18 hectorite | | | | | | | | | | | |
| | and propylene carbonate | | | | | | | | | | | |
| | glyceryl stearate and | | | | | | | | | | | |
| | cetyl alcohol | | | | | | | | | | | |
| alpha-Bisabolol | Bisabolol | 0.1 | | 0.1 | | 0.2 | 0.1 | 0.1 | 0.1 | | | |
| (Symrise) | | | | | | | | | | | | |
| 1,3-Butylene | 1,3-Butylene glycol | | | 3.0 | | | | | | | | |
| glycol | | | | | | | | | | | | |
| Carbopol 2050 ® | Carbomer | | | 0.2 | | | 0.1 | | | | | |
| (B. F. Goodrich) | | | | | | | | | | | | |
| Carbopol ETD | Carbomer | | | | | 0.5 | | | | | | |
| 2001 (Noveon) | | | | | | | | | | | | |
| Ceramide 2 | Ceramide 2 | | | | | | | | | | 0.1 | |
| (Sederma) | | | | | | | | | | | | |
| Ceramide SL | Hydroxyethyl palmityl | | | | | | | | | | | 0.1 |
| (Sino Lion) | oxyhydroxypropyl | | | | | | | | | | | |
| | palmitamide | | | | | | | | | | | |
| Cetiol SN ® | Cetyl and stearyl | 5.0 | 4.0 | 5.0 | | | | | | | | |
| (Cognis) | isononanoate | | | | | | | | | | | |
| Cetiol OE (Cognis) | Dicaprylyl ether | | | | | | | 3.0 | | | | |
| Citric acid | Citric acid | | | | | | | | | 0.1 | | |
| Copherol 1250 ® | Tocopherol acetate | 1.0 | | 0.5 | | 0.5 | 0.5 | 0.5 | | | | |
| (Cognis) | | | | | | | | | | | | |
| Corapan TQ ® | 1,6-Diethylhexyl | | | 3.0 | | | | | | | | |
| (Symrise) | naphthalate | | | | | | | | | | | |
| Crinipan ® AD | Climbazole | | | | | | | | 0.5 | | | |
| (Symrise) | | | | | | | | | | | | |
| Crotein Q (Croda) | Hydroxypropyl | | | | | | | | | 1.0 | | |
| | trimonium, hydrolyzed | | | | | | | | | | | |
| Cutina CBS ® | Glyceryl stearate and | | 2.0 | | | | | | | | | |
| (Cognis) | cetyl alcohol and stearyl | | | | | | | | | | | |
| | alcohol and cetyl | | | | | | | | | | | |
| | palmitate and coconut | | | | | | | | | | | |
| | glyceride | | | | | | | | | | | |
| Dehymuls PGPH ® | Polyglycerol 2- | 3.0 | | | | | | | | | | |
| (Cognis) | dipolyhydroxystearate | | | | | | | | | | | |
| Dehyquart SP | Quaternium 52 | | | | | | | | | 0.5 | | |
| Dehyton K | Cocamidopropyl betaine | | | | | | | | 12.0 | | | |
| Dow Corning 193 | Dimethicone polyol | | | | 1.0 | | | | | | | |
| Fluid (Dow | | | | | | | | | | | | |
| Corning) | | | | | | | | | | | | |
| Dow Corning 200 | Dimethicone | | | | | | | | | | | |
| Fluid (Dow | | | | | | | | | | | | |
| Corning) | | | | | | | | | | | | |
| D-Panthenol | Panthenol | | | | 0.5 | | | 0.5 | 0.4 | | | |
| (BASF) | | | | | | | | | | | | |
| Dracorin 100 s.e. ® | Glyceryl stearate (and) | | 3.0 | | | | | | | | | |
| (Symrise) | PEG-100 stearate | | | | | | | | | | | |
| Dracorin CE | Glyceryl stearate citrate | | | | | | | | | | 5.0 | |
| (Symrise) | | | | | | | | | | | | |
| Dragocid Liquid | Phenoxyethanol (and) | 0.3 | 0.3 | 0.3 | | 0.3 | 0.3 | 0.3 | | 0.5 | | 0.8 |
| (Symrise) | methylparaben (and) | | | | | | | | | | | |
| | ethylparaben (and) | | | | | | | | | | | |
| | butylparaben (and) | | | | | | | | | | | |
| | propylparaben (and) | | | | | | | | | | | |
| | isobutylparaben | | | | | | | | | | | |
| Drago-Beta- | Water (aqua), butylene | | | | | | | | | | 0.3 | |
| Glucan (Symrise) | glycol, glycerin, Avena | | | | | | | | | | | |
| | sativa (oat) kernel | | | | | | | | | | | |
| | extract | | | | | | | | | | | |
| Dragoderm | Glycerin, Triticum | | | | | | | | 2.0 | | | |
| (Symrise) | vulgare (wheat) gluten, | | | | | | | | | | | |
| | water (aqua) | | | | | | | | | | | |
| Dragophos S | Sodium dihydroxycetyl | | | | | | | | | | | |
| (Symrise) | phosphate | | | | | | | | | | | |
| Dragorin GMS | Glyceryl stearate | | | 2.0 | | | 2.0 | | | | | |
| (Symrise) | | | | | | | | | | | | |
| Dragosan W/O | Polyglyceryl-3 | | | | | | | | | | | 1.0 |
| Liquid (Symrise) | polyricinoleate, sorbitan | | | | | | | | | | | |
| | isostearate | | | | | | | | | | | |
| Dragosan W/O P | Sorbitan isostearate, | | | | | | | | | | | 6.0 |
| (Symrise) | hydrogenated castor oil, | | | | | | | | | | | |
| | ceresin, beeswax (Cera | | | | | | | | | | | |
| | alba) | | | | | | | | | | | |
| Dragoxat EH | Ethylhexyl | | | | | | | | | | 3.0 | |
| (Symrise) | Ethyl hexanoate | | | | | | | | | | | |
| Edeta BD ® | Disodium EDTA | 0.1 | 0.1 | 0.1 | | 0.1 | 0.1 | 0.1 | | | | |
| (BASF) | | | | | | | | | | | | |
| Emulgin B2 ® | Ceteareth-20 | | 1.0 | | | | | | | 0.7 | | |
| (Cognis) | | | | | | | | | | | | |
| Emulsiphos | Cetyl phosphate, | | | 1.5 | | | 1.5 | | | | | |
| (Symrise) | hydrogenated palm | | | | | | | | | | | |
| | glycerides | | | | | | | | | | | |
| Ethanol (96%) | Ethyl alcohol | | | | 13.0 | 5.0 | | | | | | |
| Euxyl K 100 ® | Methylchlorisothiazolinone, | | | | 0.1 | | | | | | | |
| (Schülke & Mayr) | methyisothiazolinone | | | | | | | | | | | |
| Extrapon Aloe | Aqua, Aloe barbadensis, | | | | 1.0 | | | | | | | |
| vera (Symrise) | propylene glycol, alcohol | | | | | | | | | | | |
| Extrapon | Glycerin, water (aqua), | | | | 1.0 | | | | | | | |
| chamomile | Chamomilla recutita | | | | | | | | | | | |
| (Symrise) | (Matricaria) flower | | | | | | | | | | | |
| | extract | | | | | | | | | | | |
| Extrapon witch | Propylene glycol, | | | | 1.0 | | | | | | | |
| hazel (Symrise) | Hamamelis virginiana | | | | | | | | | | | |
| | (witch hazel) water, | | | | | | | | | | | |
| | water (aqua), alcohol, | | | | | | | | | | | |
| | Hamamelis virginiana | | | | | | | | | | | |
| | (witch hazel) | | | | | | | | | | | |
| | bark/leaf/twig extract | | | | | | | | | | | |
| Glycerol 85% | Glycerin | | | | | | | | | | 3.0 | 2.0 |
| Glycerol 99% | Glycerin | 4.0 | 3.0 | | 4.5 | | 3.0 | 4.0 | | | | |
| Hydrolite-5 | 1,2-Pentanediol | | | | | 4.0 | | 5.0 | | | | |
| (Symrise) | | | | | | | | | | | | |
| Isodragol | Triisononanoin | | | | | | | | | | | |
| (Symrise) | | | | | | | | | | | | |
| Isopropyl myristate | Isopropyl myristate | | | | | | | | | | | |
| (Symrise) | | | | | | | | | | | | |
| Isopropyl palmitate | Isopropyl palmitate | | | | | | | | | | 4.0 | |
| (Symrise) | | | | | | | | | | | | |
| Karion F (Merck) | Sorbitol | | | | | | | | | | | 2.0 |
| Keltrol RD (CP- | Xanthan gum | | | | | | | | | | 0.2 | |
| Kelco) | | | | | | | | | | | | |
| Keltrol T ® | Xanthan gum | | | 0.2 | 0.2 | 0.3 | | | | | | |
| (Calgon) | | | | | | | | | | | | |
| Kojic acid | Kojic acid | | | | | | | | | | 1.0 | |
| (Cosmetochem) | | | | | | | | | | | | |
| Lanette E ® | Sodium cetearyl sulfate | | | 0.7 | | | | | | | | |
| (Cognis) | | | | | | | | | | | | |
| Lanette O | Cetyl and stearyl alcohol | | 1.1 | | | | | | | 2.5 | | |
| (Cognis) | | | | | | | | | | | | |
| Lanette 16 | Cetyl alcohol | | | 1.2 | | | 0.5 | | | | 1.0 | |
| (Cognis) | | | | | | | | | | | | |
| Lanette 18 (Care | Stearyl alcohol | | | | | | | | | | 4.5 | |
| Chemicals) | | | | | | | | | | | | |
| Lara Care A-200 | Galactoarabinan | | | | | | | 0.2 | | | | |
| (Rahn) | | | | | | | | | | | | |
| Mg ascorbyl | Magnesium ascorbyl | | | | | | | | | | 3.0 | |
| phosphate | phosphate | | | | | | | | | | | |
| Magnesium | Magnesium chloride | | | | | | | | | | | 0.7 |
| chloride (Merck) | | | | | | | | | | | | |
| Monomuls 90-O | Glyceryl oleate | 1.0 | | | | | | | | | | |
| 18 ® | | | | | | | | | | | | |
| (Cognis) | | | | | | | | | | | | |
| Myritol 318 ® | Caprylic/capric acid | 6.0 | 5.0 | | | | | | | | | |
| (Cognis) | triglyceride | | | | | | | | | | | |
| NaOH 10% aq. | Sodium hydroxide | | | 2.8 | | 2.2 | 2.9 | 0.6 | | | | |
| solution | | | | | | | | | | | | |
| Sodium ascorbyl | Sodium ascorbyl | | | | | | | | | | 2.0 | |
| phosphate (EMD | phosphate | | | | | | | | | | | |
| Chemicals) | | | | | | | | | | | | |
| Natrosol 250 HHR | Hydroxymethyl cellulose | | 0.3 | | | | | | | | | |
| (Aqualon) | | | | | | | | | | | | |
| Neo-Dragocid | Methylparaben, sorbic | | | | | | | | | | | |
| powder (Symrise) | acid, dehydroacetic acid, | | | | | | | | | | | |
| | propylparaben | | | | | | | | | | | |
| Neo Heliopan ® AP | Disodium | 10.0 | | 22.0 | | | | | | | | |
| (Symrise), 15% as | phenyldibenzimidazole | | | | | | | | | | | |
| sodium salt | tetrasulfonate | | | | | | | | | | | |
| Neo Heliopan ® AP | Disodium | | | | 22.0 | | | | | | | |
| (Symrise), (10% | phenyldibenzimidazole | | | | | | | | | | | |
| aq. solution | tetrasulfonate | | | | | | | | | | | |
| neutralized with | | | | | | | | | | | | |
| NaOH) | | | | | | | | | | | | |
| Neo Heliopan ® AV | Ethylhexyl | 4.0 | | | | 5.0 | 6.0 | 2.0 | | | | |
| (Symrise) | methoxycinnamate | | | | | | | | | | | |
| Neo Heliopan ® BB | Benzophenone-3 | | 1.0 | | | | | | | | | |
| (Symrise) | | | | | | | | | | | | |
| Neo Heliopan ® | Octocrylene | | 7.0 | | | | | | | | | |
| 303 (Symrise) | | | | | | | | | | | | |
| Neo Heliopan ® | Butyl methoxydibenzoyl- | | | 2.0 | | 1.5 | 1.5 | 1.5 | 0.5 | 0.5 | | |
| 357 (Symrise) | methane | | | | | | | | | | | |
| Neo Heliopan ® | Isoamyl p- | 4.0 | | | | 5.0 | | 6.0 | | 2.0 | | |
| E 1000 (Symrise) | methoxycinnamate | | | | | | | | | | | |
| Neo Heliopan ® | Homosalate | | | 5.0 | | | | | | | | |
| HMS (Symrise) | | | | | | | | | | | | |
| Neo Heliopan ® | Phenylbenzimidazole | | | | 33.3 | 10.0 | 13.3 | | 3.3 | | | |
| Hydro | sulfonic acid | | | | | | | | | | | |
| (15% aq. solution | | | | | | | | | | | | |
| neutralized with | | | | | | | | | | | | |
| NaOH) (Symrise) | | | | | | | | | | | | |
| Neo Heliopan ® | Menthyl anthranilate | | 3.0 | | | | | | | | | |
| MA (Symrise) | | | | | | | | | | | | |
| Neo Heliopan ® | 4-Methylbenzylidene | 2.0 | | | | 2.0 | 4.0 | 3.0 | | | | |
| MBC | camphor | | | | | | | | | | | |
| (Symrise) | | | | | | | | | | | | |
| Neo Heliopan ® | Ethylhexyl salicylate | 3.0 | | | | | | | | | | |
| OS | | | | | | | | | | | | |
| (Symrise) | | | | | | | | | | | | |
| Neutral oil | Caprylic/capric acid | | | 5.0 | | | 2.0 | | | | 6.0 | |
| (Symrise) | triglyceride | | | | | | | | | | | |
| Octyl triazone | Ethylhexyl triazone | 1.0 | | | | | | | | | | |
| Oxynex 2004 | BHT | | | | | | | | | | | 0.1 |
| (Merck) | | | | | | | | | | | | |
| Paraffin oil 5 E | Paraffinum liquidum | | | | | | | | | | | |
| (Parafluid) | | | | | | | | | | | | |
| Perfume oil | Parfum (fragrance) | 0.3 | 0.3 | 0.3 | | 0.3 | 0.4 | 0.2 | 0.5 | 0.4 | 0.3 | 0.4 |
| (Symrise | | | | | | | | | | | | |
| Fragrance) | | | | | | | | | | | | |
| PCL Liquid | Cetearyl ethylhexanoate, | | | | | | | | | | | 12.0 |
| (Symrise) | isopropyl myristate | | | | | | | | | | | |
| PCL Liquid 100 | Cetearyl ethylhexoate | | | | | | | | | | 3.0 | |
| (Symrise) | | | | | | | | | | | | |
| Pemulen TR 2 | Acrylates/C10-30 alkyl | | | | | | | 0.2 | | | | |
| (Novion) | acrylate crosspolymer | | | | | | | | | | | |
| Permulgin 2550 ® | Beeswax | 1.0 | | | | | | | | | | |
| (Koster Keunen) | | | | | | | | | | | | |
| Phenoxyethanol | Phenoxyethanol | 0.7 | | 0.7 | | 0.7 | 0.7 | 0.7 | | | | |
| (Symrise) | | | | | | | | | | | | |
| Polymer JR 400 | Polyquaternium-10 | | | | | | | | 0.4 | | | |
| 1,2-Propylene | Propylene Glycol | | | | | | | | | | | |
| glycol | | | | | | | | | | | | |
| Retinyl palmitate | Retinyl palmitate | | | | | | | | | | | 0.2 |
| in oil (DSM | | | | | | | | | | | | |
| Nutritional | | | | | | | | | | | | |
| Products) | | | | | | | | | | | | |
| Softigen 767 | PEG-6 caprylic/capric | | | | | | | | 2.5 | | | |
| | glycerides | | | | | | | | | | | |
| Solubilizer | PEG 40 hydrogenated | | | | | | | | 3.0 | | | |
| (Symrise) | castor oil, trideceth-9, | | | | | | | | | | | |
| | propylene glycol, water | | | | | | | | | | | |
| Sunflower oil | Helianthus annuus | | | | | | | | | | | 5.0 |
| (Wagner) | (sunflower) seed oil | | | | | | | | | | | |
| Sweet almond oil | Prunus dulcis | | | | | | | | | | | 5.0 |
| (Wagner) | | | | | | | | | | | | |
| SymCalmin | Butylene glycol, | | | 0.5 | | | | | | | | |
| | pentylene glycol, | | | | | | | | | | | |
| | hydroxyphenyl | | | | | | | | | | | |
| | propamidobenzoic acid | | | | | | | | | | | |
| Symdiol 68 | 1,2-Hexanediol, caprylyl | | | 0.5 | | | | | | | 0.5 | |
| (Symrise) | glycol | | | | | | | | | | | |
| SymMatrix | Maltodextrin, Rubus | | | | | | | | | | | 1.0 |
| (Symrise) | fruticosus (blackberry) | | | | | | | | | | | |
| | leaf extract | | | | | | | | | | | |
| SymWhite 377 | 4-(1-Phenylethyl)-1,3- | | | | | | | | | | 0.5 | |
| | benzenediol | | | | | | | | | | | |
| Tegosoft TN ® | C12-C15 alkyl benzoate | 6.0 | | | | 4.0 | 2.0 | | | | | |
| (Goldschmidt) | | | | | | | | | | | | |
| Texapon N 70 | Sodium laureth sulfate | | | | | | 0.1 | 0.5 | | | | |
| (Cognis) | | | | | | | | | | | | |
| Texapon NSO BZ | Sodium laureth sulfate | | | | | | | | 27.0 | | | |
| (Cognis) | | | | | | | | | | | | |
| Titanium dioxide, | Titanium dioxide | | 5.0 | | | | | | | | | |
| microfine | | | | | | | | | | | | |
| Tocopherol | Tocopherol acetate | | | | | | | | | | | 3.0 |
| acetate (DSM | | | | | | | | | | | | |
| Nutritional | | | | | | | | | | | | |
| Products) | | | | | | | | | | | | |
| Unimer U-151 | PVP/hexadecene | | | | | 0.5 | | | | | | |
| (Induchem) | copolymer | | | | | | | | | | | |
| Veegum ultra ® | Magnesium aluminum | | 1.0 | | | | | | | | | |
| (Vanderbilt) | sulfate | | | | | | | | | | | |
| Witch hazel | Hamamelis virginiana | | | | | | | | 1.0 | | | |
| distillate (Symrise) | (witch hazel) | | | | | | | | | | | |
| Zinc oxide neutral | Zinc oxide | 7.0 | | | | | | | | | | |
| (Symrise) | | | | | | | | | | | | |
| Water, dist. | Aqua (water) | to | to | to | to | to | to | to | to | to | to | to |
| | | make | make | make | make | make | make | make | make | make | make | make |
| | | up to | up to | up to | up to | up to | up to | up to | up to | up to | up to | up to |
| | | 100 | 100 | 100 | 100 | 100 | 100 | 100 | 100 | 100 | 100 | 100 |

### EXAMPLE 6

### Examples of Formulations for skin-lightening Agents

Key to Table 3 below:

Formulation 1: “Water in oil” emulsion with UVA/B broadband protection

Formulation 2: “Oil in water” emulsion with UVA/B broadband protection

Formulation 3: Skin-lightening “oil in water” emulsion with UVA/B broadband protection

Formulation 4: Skin-lightening oil-free sunspray with UVA/B broadband protection

Formulation 5: Skin-lightening balm with UVA/UVB protection

Formulation 6: Skin-lightening aerosol foam with UVB/UVA protection

Formulation 7: Skin-lightening nonaerosol foam

Formulation 8: Shampoo with hair-lightening characteristics

Formulation 9: Hair-lightening hair conditioner with UVB/UVA protection

Formulation 10: Skin-lightening O/W moisture cream

Formulation 11: Skin-lightening O/W face cream

TABLE 3
Compositions of formulations according to the invention (formulations 1-11)

| RAW MATERIAL | | | | | | | | | | | | |
|---|---|---|---|---|---|---|---|---|---|---|---|---|
| NAME | | Weight % | | | | | | | | | | |
| (MANUFACTURER) | INCI | 1 | 2 | 3 | 4 | 5 | 6 | 7 | 8 | 9 | 10 | 11 |
| Skin-lightening | | | | | | | | | | | | |
| agent | | | | | | | | | | | | |
| 3-Methoxy-4- | | 0.01 | 5.0 | 0.05 | 0.2 | 1.0 | 0.5 | 0.1 | 0.5 | 0.2 | 1.0 | 0.5 |
| nitroflavone | | | | | | | | | | | | |
| SymWhite377 | Phenylethyl resorcinol | | 0.5 | | | | | 0.1 | | | | |
| beta-Arbutin, | Arbutin | 1.0 | | | | 0.5 | | | | | 0.2 | |
| Nicotinamide | Niacinamide | | | | 0.5 | | | | | | 1.0 | |
| Kojic acid | Kojic acid | | | 0.5 | | | | | | | | 1.0 |
| Further ingredients | | | | | | | | | | | | |
| Abil 100 ® | Dimethicone | | 1.0 | 0.3 | | | | | | | | 0.3 |
| (Goldschmidt) | | | | | | | | | | | | |
| Dracorin 100 s.e. ® | Glyceryl stearate (and) | | 3.0 | | | | | | | | | |
| (Symrise) | PEG-100 stearate | | | | | | | | | | | |
| Arlypon F | Laureth-2 | | | | | | | | 2.0 | | | |
| Baysilone Oil M10 | Dimethicone | | | | | | | 1.0 | | | | |
| (GE Bayer) | | | | | | | | | | | | |
| Baysilone Oil PK | Phenyl trimethicone | | | | | 5.0 | | | | | | |
| 20 (GE Bayer) | | | | | | | | | | | | |
| Bentone Gel M | Mineral oil and | | 3.0 | | | | | | | | | |
| IO ® (Rheox) | quaternium-18 hectorite | | | | | | | | | | | |
| | and propylene carbonate | | | | | | | | | | | |
| | glyceryl stearate and | | | | | | | | | | | |
| | cetyl alcohol | | | | | | | | | | | |
| alpha-Bisabolol | Bisabolol | 0.1 | | 0.1 | | 0.2 | 0.1 | 0.1 | 0.1 | | | |
| (Symrise) | | | | | | | | | | | | |
| 1,3-Butylene | 1,3-Butylene glycol | | | 3.0 | | | | | | | | |
| glycol | | | | | | | | | | | | |
| Carbopol 2050 ® | Carbomer | | | 0.2 | | | 0.1 | | | | | |
| (B. F. Goodrich) | | | | | | | | | | | | |
| Carbopol ETD | Carbomer | | | | | 0.5 | | | | | | |
| 2001 (Noveon) | | | | | | | | | | | | |
| Cetiol SN ® | Cetyl and stearyl | 5.0 | 4.0 | 5.0 | | | | | | | | |
| (Cognis) | isononanoate | | | | | | | | | | | |
| Cetiol OE (Cognis) | Dicaprylyl ether | | | | | | | 3.0 | | | | |
| Citric acid | Citric acid | | | | | | | | | 0.1 | 0.3 | |
| Copherol 1250 ® | Tocopherol acetate | 1.0 | | 0.5 | | 0.5 | 0.5 | 0.5 | | | | |
| (Cognis) | | | | | | | | | | | | |
| Corapan TQ ® | 1,6-Diethylhexyl | | | 3.0 | | | | | | | | |
| (Symrise) | naphthalate | | | | | | | | | | | |
| Crinipan ® AD | Climbazole | | | | | | | | 0.5 | | | |
| (Symrise) | | | | | | | | | | | | |
| Crotein Q (Croda) | Hydroxypropyl | | | | | | | | | 1.0 | | |
| | trimonium, hydrolyzed | | | | | | | | | | | |
| Cutina CBS ® | Glyceryl stearate and | | 2.0 | | | | | | | | | |
| (Cognis) | cetyl alcohol and stearyl | | | | | | | | | | | |
| | alcohol and cetyl | | | | | | | | | | | |
| | palmitate and coconut | | | | | | | | | | | |
| | glyceride | | | | | | | | | | | |
| Dehymuls PGPH ® | Polyglycerol 2- | 3.0 | | | | | | | | | | |
| (Cognis) | dipolyhydroxystearate | | | | | | | | | | | |
| Dehyquart SP | Quaternium 52 | | | | | | | | | 0.5 | | |
| Dehyton K | Cocamidopropyl betaine | | | | | | | | 12.0 | | | |
| Dow Corning 193 | Dimethicone polyol | | | | 1.0 | | | | | | | |
| Fluid (Dow | | | | | | | | | | | | |
| Corning) | | | | | | | | | | | | |
| Dow Corning 200 | Dimethicone | | | | | | | | | | 2.0 | |
| Fluid (Dow | | | | | | | | | | | | |
| Corning) | | | | | | | | | | | | |
| D-Panthenol | Panthenol | | | | 0.5 | | | 0.5 | 0.4 | | | |
| (BASF) | | | | | | | | | | | | |
| Dragocid Liquid | Phenoxyethanol (and) | 0.3 | 0.3 | 0.3 | | 0.3 | 0.3 | 0.3 | | 0.5 | 0.8 | |
| (Symrise) | methylparaben (and) | | | | | | | | | | | |
| | ethylparaben (and) | | | | | | | | | | | |
| | butylparaben (and) | | | | | | | | | | | |
| | propylparaben (and) | | | | | | | | | | | |
| | isobutylparaben | | | | | | | | | | | |
| Dragophos S | Sodium dihydroxycetyl | | | | | | | | | | 2.0 | |
| (Symrise) | phosphate | | | | | | | | | | | |
| Dragorin GMS | Glyceryl stearate | | | 2.0 | | | 2.0 | | | | 2.0 | 3.0 |
| (Symrise) | | | | | | | | | | | | |
| Dracorin 100 s.e. | Glyceryl stearate, PEG- | | | | | | | | | | | 8.0 |
| P (Symrise) | 100 stearate | | | | | | | | | | | |
| Edeta BD ® | Disodium EDTA | 0.1 | 0.1 | 0.1 | | 0.1 | 0.1 | 0.1 | | | | |
| (BASF) | | | | | | | | | | | | |
| Emulgin B2 ® | Ceteareth-20 | | 1.0 | | | | | | | 0.7 | | |
| (Cognis) | | | | | | | | | | | | |
| Emulsiphos | Cetyl phosphate, | | | 1.5 | | | 1.5 | | | | | |
| (Symrise) | hydrogenated palm | | | | | | | | | | | |
| | glycerides | | | | | | | | | | | |
| Ethanol (96%) | Ethyl alcohol | | | | 13.0 | 5.0 | | | | | | |
| Euxyl K 100 ® | Methylchlorisothiazolinone, | | | | 0.1 | | | | | | | |
| (Schülke & Mayr) | methyisothiazolinone | | | | | | | | | | | |
| Extrapon Aloe | Aqua, Aloe barbadensis, | | | | 1.0 | | | | | | | |
| vera (Symrise) | propylene glycol, alcohol | | | | | | | | | | | |
| Extrapon | Glycerin, water (aqua), | | | | 1.0 | | | | | | | |
| chamomile | Chamomilla recutita | | | | | | | | | | | |
| (Symrise) | (Matricaria) flower | | | | | | | | | | | |
| | extract | | | | | | | | | | | |
| Extrapon witch | Propylene glycol, | | | | 1.0 | | | | | | | |
| hazel (Symrise) | Hamamelis virginiana | | | | | | | | | | | |
| | (witch hazel) water, | | | | | | | | | | | |
| | water (aqua), alcohol, | | | | | | | | | | | |
| | Hamamelis virginiana | | | | | | | | | | | |
| | (witch hazel) | | | | | | | | | | | |
| | bark/leaf/twig extract | | | | | | | | | | | |
| Glycerol 99% | Glycerin | 4.0 | 3.0 | | 4.5 | | 3.0 | 4.0 | | | | |
| Hydrolite-5 | 1,2-Pentanediol | | | | | 4.0 | | 5.0 | | | 3.0 | |
| (Symrise) | | | | | | | | | | | | |
| Isodragol | Triisononanoin | | | | | | | | | | 7.0 | |
| (Symrise) | | | | | | | | | | | | |
| Isopropyl myristate | Isopropyl myristate | | | | | | | | | | | 8.0 |
| (Symrise) | | | | | | | | | | | | |
| Keltrol T ® | Xanthan gum | | | 0.2 | 0.2 | 0.3 | | | | | | |
| (Calgon) | | | | | | | | | | | | |
| Lanette E ® | Sodium cetearyl sulfate | | | 0.7 | | | | | | | | |
| (Cognis) | | | | | | | | | | | | |
| Lanette O | Cetyl and stearyl alcohol | | 1.1 | | | | | | | 2.5 | | |
| (Cognis) | | | | | | | | | | | | |
| Lanette 16 | Cetyl alcohol | | | 1.2 | | | 0.5 | | | | | 2.0 |
| (Cognis) | | | | | | | | | | | | |
| Lanette 18 (Care | Stearyl alcohol | | | | | | | | | | 4.5 | |
| Chemicals) | | | | | | | | | | | | |
| Lara Care A-200 | Galactoarabinan | | | | | | | 0.2 | | | | |
| (Rahn) | | | | | | | | | | | | |
| Mg ascorbyl | Magnesium ascorbyl | | | | | | | | | | 3.0 | |
| phosphate | phosphate | | | | | | | | | | | |
| Monomuls 90-O | Glyceryl oleate | 1.0 | | | | | | | | | | |
| 18 ® | | | | | | | | | | | | |
| (Cognis) | | | | | | | | | | | | |
| Myritol 318 ® | Caprylic/capric acid | 6.0 | 5.0 | | | | | | | | | |
| (Cognis) | triglyceride | | | | | | | | | | | |
| NaOH 10% aq. | Sodium hydroxide | | | 2.8 | | 2.2 | 2.9 | 0.6 | | | | 0.2 |
| solution | | | | | | | | | | | | |
| Natrosol 250 HHR | Hydroxymethyl cellulose | | 0.3 | | | | | | | | | |
| (Aqualon) | | | | | | | | | | | | |
| Neo-Dragocid | Methylparaben, sorbic | | | | | | | | | | | 0.8 |
| powder (Symrise) | acid, dehydroacetic acid, | | | | | | | | | | | |
| | propylparaben | | | | | | | | | | | |
| Neo Heliopan ® AP | Disodium | 10.0 | | 22.0 | | | | | | | | |
| (Symrise), 15% as | phenyldibenzimidazole | | | | | | | | | | | |
| sodium salt | tetrasulfonate | | | | | | | | | | | |
| Neo Heliopan ® AP | Disodium | | | | 22.0 | | | | | | | |
| (Symrise), (10% | phenyldibenzimidazole | | | | | | | | | | | |
| aq. solution | tetrasulfonate | | | | | | | | | | | |
| neutralized with | | | | | | | | | | | | |
| NaOH) | | | | | | | | | | | | |
| Neo Heliopan ® AV | Ethylhexyl | 4.0 | | | | 5.0 | 6.0 | 2.0 | | | | |
| (Symrise) | methoxycinnamate | | | | | | | | | | | |
| Neo Heliopan ® BB | Benzophenone-3 | | 1.0 | | | | | | | | | |
| (Symrise) | | | | | | | | | | | | |
| Neo Heliopan ® | Octocrylene | | 7.0 | | | | | | | | | |
| 303 (Symrise) | | | | | | | | | | | | |
| Neo Heliopan ® | Butyl methoxydibenzoyl- | | | 2.0 | | 1.5 | 1.5 | 1.5 | 0.5 | 0.5 | | |
| 357 (Symrise) | methane | | | | | | | | | | | |
| Neo Heliopan ® | Isoamyl p-methoxy- | 4.0 | | | | 5.0 | | 6.0 | | 2.0 | | |
| E 1000 (Symrise) | cinnamate | | | | | | | | | | | |
| Neo Heliopan ® | Homosalate | | | 5.0 | | | | | | | | |
| HMS (Symrise) | | | | | | | | | | | | |
| Neo Heliopan ® | Phenylbenzimidazole | | | | 33.3 | 10.0 | 13.3 | | 3.3 | | | |
| Hydro | sulfonic acid | | | | | | | | | | | |
| (15% aq. solution | | | | | | | | | | | | |
| neutralized with | | | | | | | | | | | | |
| NaOH) (Symrise) | | | | | | | | | | | | |
| Neo Heliopan ® | Menthyl anthranilate | | 3.0 | | | | | | | | | |
| MA (Symrise) | | | | | | | | | | | | |
| Neo Heliopan ® | 4-Methylbenzylidene | 2.0 | | | | 2.0 | 4.0 | 3.0 | | | | |
| MBC | camphor | | | | | | | | | | | |
| (Symrise) | | | | | | | | | | | | |
| Neo Heliopan ® | Ethylhexyl salicylate | 3.0 | | | | | | | | | | |
| OS | | | | | | | | | | | | |
| (Symrise) | | | | | | | | | | | | |
| Neutral oil | Caprylic/capric acid | | | 5.0 | | | 2.0 | | | | | |
| (Symrise) | triglyceride | | | | | | | | | | | |
| Octyl triazone | Ethylhexyl triazone | 1.0 | | | | | | | | | | |
| Paraffin oil 5 E | Paraffinum liquidum | | | | | | | | | | | 4.0 |
| (Parafluid) | | | | | | | | | | | | |
| Perfume oil | Parfum (fragrance) | 0.3 | 0.3 | 0.3 | | 0.3 | 0.4 | 0.2 | 0.5 | 0.4 | 0.3 | 0.3 |
| PCL Liquid | Cetearyl ethylhexanoate, | | | | | | | | | | 3.0 | |
| (Symrise) | isopropyl myristate | | | | | | | | | | | |
| Pemulen TR 2 | Acrylates/C10-30 alkyl | | | | | | | 0.2 | | | | |
| (Novion) | acrylate crosspolymer | | | | | | | | | | | |
| Permulgin 2550 ® | Beeswax | 1.0 | | | | | | | | | | |
| (Koster Keunen) | | | | | | | | | | | | |
| Phenoxyethanol | Phenoxyethanol | 0.7 | | 0.7 | | 0.7 | 0.7 | 0.7 | | | | |
| (Symrise) | | | | | | | | | | | | |
| Polymer JR 400 | Polyquaternium-10 | | | | | | | | 0.4 | | | |
| 1,2-Propylene | Propylene Glycol | | | | | | | | | | | 5.0 |
| glycol | | | | | | | | | | | | |
| Softigen 767 | PEG-6 caprylic/capric | | | | | | | | 2.5 | | | |
| | glycerides | | | | | | | | | | | |
| Solubilizer | PEG 40 hydrogenated | | | | | | | | 3.0 | | | |
| (Symrise) | castor oil, trideceth-9, | | | | | | | | | | | |
| | propylene glycol, water | | | | | | | | | | | |
| Symdiol 68 | 1,2-Hexanediol, caprylyl | | | 0.5 | | | | | | | | 1.0 |
| (Symrise) | glycol | | | | | | | | | | | |
| Tegosoft TN ® | C12-C15 alkyl benzoate | 6.0 | | | | 4.0 | 2.0 | | | | | |
| (Goldschmidt) | | | | | | | | | | | | |
| Texapon N 70 | Sodium laureth sulfate | | | | | | 0.1 | 0.5 | | | | |
| (Cognis) | | | | | | | | | | | | |
| Texapon NSO BZ | Sodium laureth sulfate | | | | | | | | 27.0 | | | |
| (Cognis) | | | | | | | | | | | | |
| Titanium dioxide, | Titanium dioxide | | 5.0 | | | | | | | | | |
| microfine | | | | | | | | | | | | |
| Unimer U-151 | PVP/hexadecene | | | | | 0.5 | | | | | | |
| (Induchem) | copolymer | | | | | | | | | | | |
| Veegum ultra ® | Magnesium aluminum | | 1.0 | | | | | | | | | |
| (Vanderbilt) | sulfate | | | | | | | | | | | |
| Witch hazel | Hamamelis virginiana | | | | | | | | 1.0 | | | |
| distillate (Symrise) | (witch hazel) | | | | | | | | | | | |
| Zinc oxide neutral | Zinc oxide | 7.0 | | | | | | | | | | |
| (Symrise) | | | | | | | | | | | | |
| Water, dist. | Aqua (water) | to | to | to | to | to | to | to | to | to | to | to |
| | | make | make | make | make | make | make | make | make | make | make | make |
| | | up to | up to | up to | up to | up to | up to | up to | up to | up to | up to | up to |
| | | 100 | 100 | 100 | 100 | 100 | 100 | 100 | 100 | 100 | 100 | 100 |

### EXAMPLE 7

### Examples of Formulations for Skin-Tanning Agent

Key to Table 4 below:
Formulation 1: Skin-tanning “water in oil” emulsion with UVA/B broadband protection
Formulation 2: Intensive skin-tanning “oil in water” emulsion with UVA/B broadband protection
Formulation 3: Skin-tanning “water in oil” emulsion
Formulation 4: Skin-tanning “oil in water” emulsion with UVA/B broadband protection
Formulation 5: Skin-tanning “oil in water” cream
Formulation 6: Skin-tanning aerosol foam with UVB/UVA protection
Formulation 7: O/W self-tanning cream
Formulation 8: Shampoo with skin- and hair-lightening characteristics
Formulation 9: Skin- and hair-lightening conditioner with UVB/UVA protection
Formulation 10: Skin-tanning O/W moisture cream
Formulation 11: Skin-tanning O/W face cream

| RAW MATERIAL NAME | | Weight % | | | | | | | | | | |
|---|---|---|---|---|---|---|---|---|---|---|---|---|
| (MANUFACTURER) | INCI | 1 | 2 | 3 | 4 | 5 | 6 | 7 | 8 | 9 | 10 | 11 |
| AhR agonist | | | | | | | | | | | | |
| FICZ | | 0.1 | 0.5 | 0.05 | 0.5 | 1.0 | 0.3 | 0.2 | 0.2 | 0.3 | 2.0 | 5.0 |
| Caffeine | Caffeine | 0.5 | | 0.2 | | | 0.2 | 0.1 | | | | |
| Naringin | 4′,5,7- | 0.1 | | | | | | | | 0.1 | | |
| | Trihydroxyflavone-7-O- | | | | | | | | | | | |
| | neohesperidoside | | | | | | | | | | | |
| N-acetyl tyrosine | | | 0.5 | | | | | | | | | 1.0 |
| Dihydroxyacetone | Dihydroxyacetone | | | | | | | 5.0 | | | | |
| Further ingredients | | | | | | | | | | | | |
| Abil 100 ® | Dimethicone | | 1.0 | | | 2.0 | | 0.5 | | | | 0.3 |
| (Goldschmidt) | | | | | | | | | | | | |
| Alugel 34 TH | Aluminum stearate | | | 1.0 | | | | | | | | |
| (Baerlocher) | | | | | | | | | | | | |
| Arlacel 165 ® (ICI) | Glyceryl stearate and | | 3.0 | | | | | | | | | |
| | polyethylene glycol | | | | | | | | | | | |
| | 100 stearate | | | | | | | | | | | |
| Arlypon F | Laureth-2 | | | | | | | | 2.0 | | | |
| Bentone Gel M IO ® | Mineral oil and | | 3.0 | | | | | | | | | |
| (Rheox) | quaternium-18 | | | | | | | | | | | |
| | hectorite and | | | | | | | | | | | |
| | propylene carbonate | | | | | | | | | | | |
| | glyceryl stearate and | | | | | | | | | | | |
| | cetyl alcohol | | | | | | | | | | | |
| alpha-Bisabolol | Bisabolol | 0.1 | | 0.1 | | | 0.1 | | 0.1 | | | |
| (Symrise) | | | | | | | | | | | | |
| Brij 78 ® | Steareth-20 | | 0.5 | | | | | | | | | |
| Carbopol 2050 ® | Carbomer | | | | | 0.1 | 0.1 | | | | | |
| (B. F. Goodrich) | | | | | | | | | | | | |
| Cetiol SN ® (Cognis) | Cetyl and stearyl | 5.0 | 4.0 | | | | | | | | | |
| | isononanoate | | | | | | | | | | | |
| Citric acid | Citric acid | | | | | | | | | 0.1 | 0.3 | |
| Copherol 1250 ® | Tocopherol acetate | 1.0 | | | 0.5 | | 0.5 | | | | | |
| (Cognis) | | | | | | | | | | | | |
| Corapan TQ ® | 1,6-Diethylhexyl | | | | 3.0 | | | | | | | |
| (Symrise) | naphthalate | | | | | | | | | | | |
| Crinipan ® AD | Climbazole | | | | | | | | 0.5 | | | |
| (Symrise) | | | | | | | | | | | | |
| Crotein Q (Croda) | Hydroxypropyl | | | | | | | | | 1.0 | | |
| | trimonium, hydrolyzed | | | | | | | | | | | |
| Cutina CBS ® | Glyceryl stearate and | | 2.0 | | | | | | | | | |
| (Cognis) | cetyl alcohol and | | | | | | | | | | | |
| | stearyl alcohol and | | | | | | | | | | | |
| | cetyl palmitate and | | | | | | | | | | | |
| | coconut glyceride | | | | | | | | | | | |
| Dehymuls PGPH ® | Polyglycerol 2- | 3.0 | | | | | | | | | | |
| (Cognis) | dipolyhydroxystearate | | | | | | | | | | | |
| Dehyquart SP | Quaternium 52 | | | | | | | | | 0.5 | | |
| Dehyton K | Cocamidopropyl | | | | | | | | 12.0 | | | |
| | betaine | | | | | | | | | | | |
| Dow Corning 200 | Dimethicone | | | | | | | | | | 2.0 | |
| Fluid (Dow Corning) | | | | | | | | | | | | |
| D-Panthenol (BASF) | Panthenol | | | | | | | | 0.4 | | | |
| Dragocid Liquid | Phenoxyethanol (and) | 0.3 | 0.3 | 1.0 | 0.3 | 0.8 | 0.3 | | 0.7 | 0.5 | 0.8 | |
| (Symrise) | methylparaben (and) | | | | | | | | | | | |
| | ethylparaben (and) | | | | | | | | | | | |
| | butylparaben (and) | | | | | | | | | | | |
| | propylparaben (and) | | | | | | | | | | | |
| | isobutylparaben | | | | | | | | | | | |
| Dragophos S | Sodium dihydroxycetyl | | | | | | | | | | 2.0 | |
| (Symrise) | phosphate | | | | | | | | | | | |
| Dragosan W/O P | Sorbitan isostearate, | | | 7.0 | | | | | | | | |
| (Symrise) | hydrogenated castor | | | | | | | | | | | |
| | oil, ceresin, beeswax | | | | | | | | | | | |
| | (Cera alba) | | | | | | | | | | | |
| Dracorin CE | Glyceryl stearate | | | | | | | 5.0 | | | | |
| | citrate | | | | | | | | | | | |
| Dracorin GMS | Glyceryl stearate | | | | | 2.0 | 2.0 | | | | 2.0 | 3.0 |
| (Symrise) | | | | | | | | | | | | |
| Dracorin 100 s.e. P | Glyceryl stearate, | | | | | | | | | | | 8.0 |
| (Symrise) | PEG-100 stearate | | | | | | | | | | | |
| Dragoxat EH | Ethylhexyl | | | | | 3.0 | | 3.0 | | | | |
| (Symrise) | ethylhexanoate | | | | | | | | | | | |
| Edeta BD ® (BASF) | Disodium EDTA | 0.1 | 0.1 | | 0.1 | | 0.1 | 0.1 | | | | |
| Emulgade PL | Cetearyl glucoside, | | | | | 0.5 | | | | | | |
| | cetearyl alcohol | | | | | | | | | | | |
| Emulgin B2 ® | Ceteareth-20 | | 1.0 | | | | | | | 0.7 | | |
| (Cognis) | | | | | | | | | | | | |
| Emulsiphos | Cetyl phosphate, | | | | | 2.0 | 1.5 | | | | | |
| (Symrise) | hydrogenated palm | | | | | | | | | | | |
| | glycerides | | | | | | | | | | | |
| Ethanol (96%) | Ethyl alcohol | | | | | | | 2.0 | | | | |
| Extrapon Aloe vera | Glycerin, water (aqua), | | | 3.0 | | | | | | | | |
| (Symrise) | Aloe barbadensis leaf | | | | | | | | | | | |
| | extract | | | | | | | | | | | |
| Extrapon green tea | Glycerin, water (aqua), | | | | | 0.2 | | | | | | |
| (Symrise) | Camellia sinensis leaf | | | | | | | | | | | |
| | extract | | | | | | | | | | | |
| Extrapon witch hazel | Propylene glycol, | | | 1.0 | | | | | | | | |
| (Symrise) | Hamamelis virginiana | | | | | | | | | | | |
| | (witch hazel) water, | | | | | | | | | | | |
| | water (aqua), | | | | | | | | | | | |
| | Hamamelis virginiana | | | | | | | | | | | |
| | (witch hazel) extract | | | | | | | | | | | |
| Extrapon chamomile | Glycerin, water (aqua), | | | | | 0.5 | | | | | | |
| (Symrise) | Chamomilla recutita | | | | | | | | | | | |
| | (Matricaria) flower | | | | | | | | | | | |
| | extract | | | | | | | | | | | |
| Extrapon rosemary | Glycerin, water (aqua), | | | | | 0.3 | | | | | | |
| (Symrise) | Rosmarinus officinalis | | | | | | | | | | | |
| | (rosemary) leaf extract | | | | | | | | | | | |
| Glycerol 99% | Glycerin | 4.0 | 3.0 | 2.0 | 4.0 | 2.0 | 3.0 | 1.5 | | | | |
| Hostacerin DGMS ® | Polyglceryl 2-stearate | | | | 3.0 | | | | | | | |
| (Clariant) | | | | | | | | | | | | |
| Hydrolite-5 (Symrise) | 1,2-Pentylene glycol | | | | | | | 3.5 | | | 3.0 | |
| Isodragol (Symrise) | Triisononanoin | | | | | 2.0 | | | | | 7.0 | |
| Isopropyl myristate | Isopropyl myristate | | | | | | | 4.0 | | | | 8.0 |
| (Symrise) | | | | | | | | | | | | |
| Karion F (Merck) | Sorbitol | | | 2.0 | | | | | | | | |
| Keltrol T ® (Calgon) | Xanthan gum | | | | 0.2 | 0.1 | | 0.3 | | | | |
| Lanette E ® (Cognis) | Sodium cetearyl | | | | 0.75 | | | | | | | |
| | sulfate | | | | | | | | | | | |
| Lanette O (Cognis) | Cetyl and stearyl | | 1.1 | | | 3.0 | | | | 2.5 | | |
| | alcohol | | | | | | | | | | | |
| Lanette 16 (Cognis) | Cetyl alcohol | | | | 2.0 | | 0.5 | 1.0 | | | | 2.0 |
| Lanette 18 (Care | Stearyl alcohol | | | | | | | | | | 4.5 | |
| Chemicals) | | | | | | | | | | | | |
| Mg ascorbyl | Magnesium ascorbyl | | | | | | | | | | 3.0 | |
| phosphate | phosphate | | | | | | | | | | | |
| Magnesium sulfate | Magnesium sulfate | | | 0.7 | | | | | | | | |
| heptahydrate | | | | | | | | | | | | |
| (Merck) | | | | | | | | | | | | |
| Monomuls 90-O 18 ® | Glyceryl oleate | 1.0 | | | | | | | | | | |
| (Cognis) | | | | | | | | | | | | |
| Myritol 318 ® | Caprylic/capric acid | 6.0 | 5.0 | | | 0.25 | | | | | | |
| (Cognis) | triglyceride | | | | | | | | | | | |
| NaOH 10% aq. | Sodium hydroxide | | | | 0.2 | | 2.9 | | | | | 0.2 |
| solution | | | | | | | | | | | | |
| Natrosol 250 HHR | Hydroxymethyl | | 0.3 | | | | | | | | | |
| (Aqualon) | cellulose | | | | | | | | | | | |
| Neo-Dragocid | Methylparaben, sorbic | | | | | | | | | | | 0.8 |
| powder (Symrise) | acid, dehydroacetic | | | | | | | | | | | |
| | acid, propylparaben | | | | | | | | | | | |
| Neo Heliopan ® AP | Disodium | 10.0 | | | 6.7 | | | | | | | |
| (Symrise), 15% as | phenyldibenzimidazole | | | | | | | | | | | |
| sodium salt | tetrasulfonate | | | | | | | | | | | |
| Neo Heliopan ® AV | Ethylhexyl | 4.0 | | | | | 6.0 | | | | | |
| (Symrise) | methoxycinnamate | | | | | | | | | | | |
| Neo Heliopan ® BB | Benzophenone-3 | | 1.0 | | | | | | | | | |
| (Symrise) | | | | | | | | | | | | |
| Neo Heliopan ® 303 | Octocrylene | | 7.0 | | | | | | | | | |
| (Symrise) | | | | | | | | | | | | |
| Neo Heliopan ® 357 | Butyl | | | | 0.6 | | 1.5 | | 0.5 | 0.5 | | |
| (Symrise) | methoxydibenzoylmethane | | | | | | | | | | | |
| Neo Heliopan ® | Isoamyl p- | 4.0 | | | | | | | | 2.0 | | |
| E 1000 (Symrise) | methoxycinnamate | | | | | | | | | | | |
| Neo Heliopan ® HMS | Homosalate | | | | 9.5 | | | | | | | |
| (Symrise) | | | | | | | | | | | | |
| Neo Heliopan ® | Phenylbenzimidazole | | | | 6.7 | | 13.3 | | 3.3 | | | |
| Hydro | sulfonic acid | | | | | | | | | | | |
| (15% aq. solution | | | | | | | | | | | | |
| neutralized with | | | | | | | | | | | | |
| NaOH) (Symrise) | | | | | | | | | | | | |
| Neo Heliopan ® MA | Menthyl anthranilate | | 3.0 | | | | | | | | | |
| (Symrise) | | | | | | | | | | | | |
| Neo Heliopan ® MBC | 4-Methylbenzylidene | 2.0 | | | | | 4.0 | | | | | |
| (Symrise) | camphor | | | | | | | | | | | |
| Neo Heliopan ® OS | Ethylhexyl salicylate | 3.0 | | | | | | | | | | |
| (Symrise) | | | | | | | | | | | | |
| Neutral oil (Symrise) | Caprylic/capric acid | | | | 5.0 | | 2.0 | 6.0 | | | | |
| | triglyceride | | | | | | | | | | | |
| Octyl triazone | Ethylhexyl triazone | 1.0 | | | | | | | | | | |
| Paraffin oil 5 E | Paraffinum liquidum | | | | | | | | | | | 4.0 |
| (Parafluid) | | | | | | | | | | | | |
| Perfume oil | Parfum (fragrance) | 0.3 | 0.3 | 0.4 | 0.5 | 0.3 | 0.4 | 0.3 | 0.5 | 0.4 | 0.3 | 0.3 |
| PCL Liquid | Cetearyl | | | 12.0 | | 5.0 | | 3.0 | | | 3.0 | |
| (Symrise) | ethylhexanoate, | | | | | | | | | | | |
| | isopropyl myristate | | | | | | | | | | | |
| PCL Solid (Symrise) | Stearyl heptanoate, | | | | | 2.0 | | | | | | |
| | stearyl caprylate | | | | | | | | | | | |
| Permulgin 2550 ® | Beeswax | 1.0 | | | | | | | | | | |
| (Koster Keunen, NL) | | | | | | | | | | | | |
| Phenoxyethanol | Phenoxyethanol | 0.7 | | | 0.7 | | 0.7 | | | | | |
| (Symrise) | | | | | | | | | | | | |
| Polymer JR 400 | Polyquaternium-10 | | | | | | | | 0.4 | | | |
| Prisorine 3505 ® | Isostearic acid | | | | 0.5 | | | | | | | |
| (UniQema) | | | | | | | | | | | | |
| 1,2-Propylene glycol | Propylene Glycol | | | | | 5.0 | | | | | | 5.0 |
| Sepigel 305 | Polyacrylamide, C13-14 | | | | | | | 1.0 | | | | |
| | isoparaffin, laureth-7 | | | | | | | | | | | |
| SF1214 ® (Bayer) | Cyclopentasiloxane, | | | | 1.0 | | | | | | | |
| | dimethicone | | | | | | | | | | | |
| Softigen 767 | PEG-6 caprylic/capric | | | | | | | | 2.5 | | | |
| | glycerides | | | | | | | | | | | |
| Solubilizer (Symrise) | PEG 40 hydrogenated | | | | | | | | 3.0 | | | |
| | castor oil, trideceth-9, | | | | | | | | | | | |
| | propylene glycol, water | | | | | | | | | | | |
| Sunflower Oil (H. Erhard | Helianthus annus | | | 5.0 | | | | | | | | |
| Wagner) | (sunflower) seed oil | | | | | | | | | | | |
| Sweet Almond Oil | Prunus dulcis | | | 5.0 | | | | | | | | |
| (H. Erhard Wagner) | | | | | | | | | | | | |
| Symdiol 68 | 1,2-Hexanediol, capryl | | | | 0.5 | | | | | | | |
| (Symrise) | alcohol | | | | | | | | | | | |
| SymGlucan | Water, glycerin, beta- | | | | | 0.3 | | | | | | |
| (Symrise) | glucan | | | | | | | | | | | |
| Tegosoft TN ® | C12-C15 alkyl | 6.0 | | | 2.0 | | 2.0 | | | | | |
| (Goldschmidt) | benzoate | | | | | | | | | | | |
| Texapon N 70 | Sodium laureth sulfate | | | | | | 0.1 | 0.5 | | | | |
| (Cognis) | | | | | | | | | | | | |
| Texapon NSO BZ | Sodium laureth sulfate | | | | | | | | 27.0 | | | |
| (Cognis) | | | | | | | | | | | | |
| Titanium dioxide, | Titanium dioxide | | 5.0 | | | | | | | | | |
| microfine | | | | | | | | | | | | |
| Vitamin E acetate | Tocopherol acetate | | | 3.0 | | | | | | | | |
| (DSM Nutritional | | | | | | | | | | | | |
| Products) | | | | | | | | | | | | |
| Vitamin A palmitate | Retinyl palmitate | | | 0.2 | | | | | | | | |
| in oil (1 million IU/g) | | | | | | | | | | | | |
| (DSM Nutritional | | | | | | | | | | | | |
| Products) | | | | | | | | | | | | |
| Veegum ultra ® | Magnesium aluminum | | 1.0 | | | | | | | | | |
| (Vanderbilt) | sulfate | | | | | | | | | | | |
| Witch hazel distillate | Hamamelis virginiana | | | | | | | | 1.0 | | | |
| (Symrise) | (witch hazel) | | | | | | | | | | | |
| Zinc oxide neutral | Zinc oxide | 7.0 | | | | | | | | | | |
| (Symrise) | | | | | | | | | | | | |
| Water, dist. | Aqua (water) | to | to | to | to | to | to make | to | to make | to | to | to |
| | | make | make | make | make | make | up to | make | up to | make | make | make |
| | | up to | up to | up to | up to | up to | 100 | up to | 100 | up to | up to | up to |
| | | 100 | 100 | 100 | 100 | 100 | | 100 | | 100 | 100 | 100 |

1. A method for assessing the effectiveness of an AhR agonist or AhR antagonist comprising the steps
i) stressing a cell of an ex vivo or in vitro melanocyte and/or keratinocyte cell culture or of an in vitro skin model with a possible AhR agonist or AhR antagonist,
ii) if the cell was stressed in step i) with a possible AhR antagonist, treating the stressed cell with UVB radiation or an AhR agonist, and
iii) determining induction of an AhR-inducible gene, preferably of CYP1A1.

2. The method as claimed in claim 1, furthermore comprising the steps
iv) stressing an untreated cell from step i) with a preselected compound as claimed in one of claims 10 to 13, preferably of the formula (X),
v) treating the cell stressed in step iv) with UVB radiation as in step ii),
vi) determining the induction of the AhR-inducible gene, preferably of CYP1A1, determined in step iii), and
vii) comparing the gene induction determined in step iii) and step vi).

3. The method as claimed in one of claims 1 to 2, wherein in step ii) and optionally in step v) the stressed cell(s) are treated with a polycyclic aromatic hydrocarbon and preferably with TCDD.

4. A method for screening a substance library for AhR-agonists or antagonists, comprising the steps:
1) providing a sample of each of the substances in the substance library, and
2) carrying out a method as claimed in one of claims 1 to 3 for each of the samples provided in 1),
3) selecting those substances, for the samples of which induction of the AhR-inducible gene was reduced by a preselected extent, as AhR antagonists; or
selecting those substances, for the samples of which expression of an AhR-inducible gene was increased by a preselected extent, as AhR agonists.

5. The method as claimed in claim 4, wherein the method is carried out as a high throughput method with at least 96 samples.

6. An apparatus for carrying out a high throughput screening method as claimed in claim 5, comprising:
a sample holder for at least 96 samples,
illumination means for stressing the samples with UVB radiation,
means for determining the induction of an AhR-inducible gene in one of the 96 samples, and
evaluation means for indicating induction of the AhR-inducible gene.

7. A method for producing a skin-protection preparation comprising mixing an AhR agonist and/or antagonist with a cosmetically and/or pharmaceutically acceptable carrier, such that the concentration of the AhR agonist or antagonist in the mixture amounts to at least twice the minimum concentration necessary for reducing induction of an AhR-induced gene, wherein the minimum concentration is determined with a method as claimed in one of claims 1 to 5.

8. A method for assessing the effectiveness of a skin-lightening agent or skin-tanning agent comprising the steps
i) stressing a cell with a possible skin-lightening agent or skin-tanning agent,
ii) determining the extent of melanin formation of the cell treated in step i),
iii) stressing an untreated cell from step i) with a preselected amount a) of a compound of the formula (X), if the effectiveness of a skin-lightening agent is to be assessed, and b) with formylindolo(3,2b)carbazole, if the effectiveness of a skin-tanning agent is to be assessed,
iv) determining the extent of melanin formation of the cell treated in step iii),
v) comparing the skin lightening determined in step ii) and step iv).

9. A method for producing a skin-lightening preparation comprising mixing a skin-lightening agent with a cosmetically and/or pharmaceutically acceptable carrier, such that the concentration of the skin-lightening agent in the mixture amounts to at least twice the minimum concentration necessary for skin lightening, wherein the minimum concentration is determined with a method as claimed in claim 8.

10. A compound of the formula (II) 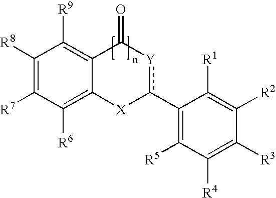
wherein
R¹ to R⁹ mutually independently mean hydrogen, hydroxy, C₁-C₁₂ alkoxy, C₁-C₁₂ alkyl, C₂-C₁₂ alkenyl, and
n=0 and Y=—CH— or —C(CH₃)—, X=O and the dashed line correspondingly means either a double bond or two hydrogens,
or
n=1, Y=O and X=methylene and the dashed line means two hydrogens.

11. The compound as claimed in claim 10, wherein R¹ to R⁹, mutually independently, mean hydrogen, hydroxy, C₁-C₄ alkoxy or C₂-C₄ alkenyl.

12. The compound as claimed in one of claims 10 to 11, wherein at least 5 of the residues R¹ to R⁹ are hydrogen.

13. The compound as claimed in one of claims 10 to 12 having the formula (X)

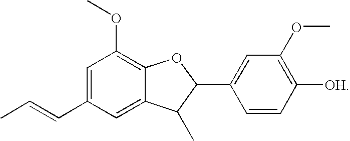

14. A preparation containing, in a pharmaceutically and/or cosmetically acceptable carrier, a compound as claimed in one of claims 10 to 13 or a compound selected by one of the methods as claimed in one of claims 1 to 5 or 8 in an sufficient amount for
(a) reducing or preventing translocation of AhR into a cell nucleus,
(b) reducing or preventing UVB-induced or UVB-inducible gene expression,
(c) reducing or preventing gene expression induced or inducible by polycyclic aromatic hydrocarbons, preferably TCDD, and/or
(d) reducing or preventing UVB-induced or UVB-inducible skin damage, in particular skin cancer, skin aging, skin inflammation and sunburn.

15. The preparation as claimed in claim 14, wherein the compound of the formula (X) is present in an amount sufficient for skin lightening.

16. The preparation as claimed in one of claims 14 to 15, containing the compound of the formula (II) in a proportion of at least 0.0001 wt. %, relative to the entire preparation.

17. The preparation as claimed in one of claims 14 to 16, furthermore containing a UVA and/or UVB filter.

18. The preparation as claimed in one of claims 14 to 17, wherein the preparation is a cosmetic preparation, preferably selected from the group consisting of a sun cream, skin-protection lotion, aftersun lotion.

19. A medicament consisting of, substantially consisting of or comprising
a compound as claimed in one of claims 10 to 13 or
a preparation as claimed in one of claims 14 to 18 or
a compound selected as claimed in one of claims 1 to 5 or 8.

20. Use of a compound as claimed in one of claims 10 to 13 for producing a medicament.

21. The use as claimed in claim 20, wherein the medicament is formulated for
(a) reducing or preventing translocation of AhR into a cell nucleus,
(b) reducing or preventing UVB-induced or UVB-inducible gene expression,
(c) reducing or preventing gene expression induced or inducible by polycyclic aromatic hydrocarbons, preferably TCDD, and/or
(d) reducing or preventing UVB-induced or UVB-inducible skin damage, in particular skin cancer, skin aging, skin inflammation and sunburn.

22. Use of a compound as claimed in one of claims 10 to 13 and/or of a compound selected by a method as claimed in one of claims 1 to 5 and 8 as an AhR antagonist, and preferably for
(a) reducing or preventing translocation of AhR into a cell nucleus,
(b) reducing or preventing UVB-induced or UVB-inducible gene expression,
(c) reducing or preventing gene expression induced or inducible by polycyclic aromatic hydrocarbons, preferably TCDD, and/or
(d) reducing or preventing UVB-induced or UVB-inducible skin damage, in particular skin cancer, skin aging, skin inflammation and sunburn.
